(19) United States
(12) Patent Application Publication
(10) Pub. No.: US 2024/0279184 A1
(43) Pub. Date: Aug. 22, 2024

(54) HETEROAROMATIC ANALOGUES OF 3-BENZYLMENADIONE DERIVATIVES AND PROCESSES FOR THEIR PREPARATION

(71) Applicants: CENTRE NATIONAL DE LA RECHERCHE SCIENTIFIQUE, PARIS (FR); UNIVERSITÉ DE STRASBOURG, STRASBOURG (FR); UNIVERSITE DE HAUTE ALSACE, MULHOUSE CEDEX (FR)

(72) Inventors: Elisabeth DAVIOUD-CHARVET, STRASBOURG (FR); Maxime DONZEL, STRASBOURG (FR); Matthieu ROIGNANT, BISCHHEIM (FR); Mourad ELHABIRI, STRASBOURG (FR)

(21) Appl. No.: 18/565,844

(22) PCT Filed: Jun. 3, 2022
(86) PCT No.: PCT/EP2022/065255

§ 371 Date: Nov. 30, 2023

(30) Foreign Application Priority Data
Jun. 4, 2021 (EP) 21305755.7

(51) Int. Cl. C07D 239/30; C07C 41/30; C07C 43/275; C07D 213/61; C07D 213/64; C07D 213/73; C07D 213/84; C07D 215/14; C07D 239/26; C07D 239/28; C07D 239/34; C07D 239/42; C07D 277/62; C07D 307/42; C07D 307/46; C07D 333/22; C07D 405/12

(52) U.S. Cl. CPC C07D 239/30; C07C 41/30; C07C 43/275; C07D 213/61; C07D 213/64; C07D 213/73; C07D 213/84; C07D 215/14; C07D 239/26; C07D 239/28; C07D 239/34; C07D 239/42; C07D 277/62; C07D 307/42; C07D 307/46; C07D 333/22; C07D 405/12

(57) ABSTRACT

A compound having the formula (I):

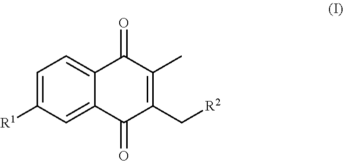

wherein:
R¹ is selected from the group consisting of: H, F, (C₁-C₆)alkoxy, and halo(C₁-C₆)alkyl; and
R² is an optionally substituted heteroaryl group, as well as processes for the preparation of the compound, and intermediate compounds.

The present invention concerns heteroaromatic analogues of 3-benzylmenadione derivatives, as well as processes for their preparation.

Unsymmetrical methylene-linked biaryl systems are important building blocks in medicinal chemistry. When applied to the 3-benzylmenadione series, no general or versatile synthetic strategy has been developed. In 1991, a team from Takeda Pharmaceutical (formerly Takeda Chemical Industry) published the synthesis of numerous quinones bearing a 3-picolinyl group. One of these was the synthesis of 3-(3-picolinyl)menadione, one of the target molecules. This team inserted a bromomethyl or chloromethyl group in position 3 to achieve a direct coupling with a lithiated 3-pyridine (Ohkawa S, Terao S, Terashita Z-1, Shibouta Y, Nishikawa K. Dual inhibitors of thromboxane A2 synthase and 5-lipoxygenase with scavenging activity of active oxygen species (AOS). Synthesis of a novel series of (3-pyridylmethyl)benzoquinone derivatives. J. Med. Chem. 1991, 34, 267-276. doi: 10.1021/jm00105a042).

In the simple di(hetero)arylmethane series, benzyl halides were efficiently coupled with various aromatic and especially heteroaromatic boronic acids by a pallado-catalysed Suzuki coupling (Henry N, Enguehard-Gueiffier C, Thery I, Gueiffier A. One-Pot Dual Substitutions of Bromobenzyl Chloride, 2-Chloromethyl-6-halogenoimidazo[1,2-a]pyridine and —[1,2-b]pyridazine by Suzuki-Miyaura Cross-Coupling Reactions. Eur. J. Org. Chem. 2008, 4824-4827. doi: 10.1002/ejoc.200800544). But, no methylene-linked biaryl systems were built within the quinone series using the Suzuki coupling reaction.

The aim of the present invention is to provide new heteroaromatic analogues of 3-benzylmenadione derivatives that could be useful as ligands or components for active oxygen barriers in plastic polymers for food and drink preservation.

Another aim of the present invention is to provide new heteroaromatic analogues of 3-benzylmenadione derivatives that could be useful as therapeutic compounds.

Another aim of the present invention is also to provide a new efficient process for the preparation of heteroaromatic analogues of 3-benzylmenadione derivatives with a satisfying yield.

The present invention thus relates to a compound having the formula (1):

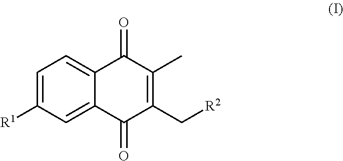

wherein:
R¹ is selected from the group consisting of: H, F, (C₁-C₆)alkoxy, and halo(C₁-C₆)alkyl; and
R² is a heteroaryl group, said heteroaryl group being optionally substituted with at least one substituent selected from the group consisting of:
   halogen,
   halo(C₁-C₆)alkyl,
   (C₁-C₆)alkoxy,
   CN,
   (C₁-C₆)alkyl,
   NRₐR_{b}, Rₐ and R_{b}, identical or different, being independently H or a (C₁-C₆)alkyl, such as NH₂,
   (C₂-C₆)alkynyl, such as —C≡C—,
   OR_{c}, R_{c} being a cycloheteroalkyl, preferably an oxetanyl group,
   NO₂,
   SF₃,
   SF₅,
   —C(═O)—(C₁-C₆)alkyl,
   halo(C₁-C₆)alkoxy, and
   (C₁-C₆)alkoxy, provided that the compound of formula (1) is different from the following compound:

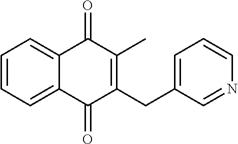 [SMILES: CC1=C(Cc2cccnc2)C(=O)c2ccccc2C1=O]

According to the invention, R² is an optionally substituted heteroaryl group as defined above being different from the following heteroaryl groups, optionally substituted:

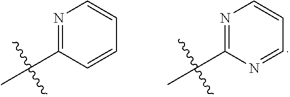 [SMILES: *c1ccccn1 ; *c1ncccn1]

According to an embodiment, R² is an optionally substituted heteroaryl group as defined above being different from the following heteroaryl groups, optionally substituted:

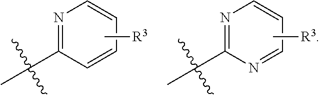

R³ being selected from the group consisting of: halogen, halo(C₁-C₆)alkyl, (C₁-C₆)alkoxy, CN, (C₁-C₆)alkyl, NRₐR_{b}, Rₐ and R_{b}, identical or different, being independently H or a (C₁-C₆)alkyl, such as NH₂, (C₂-C₆)alkynyl, such as —C≡C—, OR_{c}, R_{c} being a cycloheteroalkyl, preferably an oxetanyl group, NO₂, SF₃, SF₅, —C(═O)—(C₁-C₆)alkyl, halo(C₁-C₆)alkoxy, and (C₁-C₆)alkoxy.

The following definitions are set forth to illustrate and define the meaning and scope of the various terms used to describe the invention herein.

The expression “Cₜ-C_{z}” means a carbon-based chain which can have from t to z carbon atoms, for example C₁-C₃ means a carbon-based chain which can have from 1 to 3 carbon atoms.

The term “alkyl group” means: a linear or branched, saturated, hydrocarbon-based aliphatic group comprising, unless otherwise mentioned, from 1 to 6 carbon atoms. By way of examples, mention may be made of methyl, ethyl, n-propyl, isopropyl, butyl, isobutyl, tert-butyl or pentyl groups.

The term “aryl group” means: a cyclic aromatic group comprising between 6 and 10 carbon atoms. By way of examples of aryl groups, mention may be made of phenyl or naphthyl groups.

When an alkyl radical is substituted with an aryl group, the term “arylalkyl” or “aralkyl” radical is used. The “arylalkyl” or “aralkyl” radicals are aryl-alkyl-radicals, the aryl and alkyl groups being as defined above. Among the arylalkyl radicals, mention may in particular be made of the benzyl or phenethyl radicals.

The term “halogen” means: a fluorine, a chlorine, a bromine or an iodine.

The term “alkoxy group” means: an —O-alkyl radical where the alkyl group is as previously defined. By way of examples, mention may be made of —O—(C₁-C₄)alkyl groups, and in particular the —O-methyl group, the —O-ethyl group as —O—C₃alkyl group, the —O-propyl group, the —O-isopropyl group, and as —O—C₄alkyl group, the —O-butyl, —O-isobutyl or —O-tert-butyl group.

The term “haloalkyl group” means: an alkyl group as defined above, in which one or more of the hydrogen atoms is (are) replaced with a halogen atom. By way of example, mention may be made of fluoroalkyls, in particular CF₃ or CHF₂.

The term “haloalkoxy group” means: an —O-haloalkyl group, the haloalkyl group being as defined above. By way of example, mention may be made of fluoroalkyls, in particular OCF₃ or OCHF₂.

The abovementioned “alkyl” radicals can be substituted with one or more substituents. Among these substituents, mention may be made of the following groups: amino, hydroxyl, thiol, oxo, halogen, alkyl, alkoxy, alkylthio, alkylamino, aryloxy, arylalkoxy, cyano, trifluoromethyl, carboxy or carboxyalkyl.

The term “alkylthio” means: an —S-alkyl group, the alkyl group being as defined above.

The term “alkylamino” means: an —NH-alkyl group, the alkyl group being as defined above.

The term “aryloxy” means: an —O-aryl group, the aryl group being as defined above.

The term “arylalkoxy” means: an aryl-alkoxy-group, the aryl and alkoxy groups being as defined above.

The term “carboxyalkyl” means: an HOOC-alkyl-group, the alkyl group being as defined above. As examples of carboxyalkyl groups, mention may in particular be made of carboxymethyl or carboxyethyl.

The term “heteroalkyl group” means: an alkyl group as defined above, in which one or more of the carbon atoms is (are) replaced with a heteroatom, such as 0 or N.

The term “carboxyl” means: a COOH group.

The term “oxo” means: “═O”.

The term “heterocycloalkyl group” means: a 4- to 10-membered, saturated or partially unsaturated, monocyclic or bicyclic group comprising from one to three heteroatoms selected from O, S or N; the heterocycloalkyl group may be attached to the rest of the molecule via a carbon atom or via a heteroatom. As an example, one may cite oxetanyl.

According to an embodiment, in formula (1), R² is a heteroaryl group comprising a 5- to 10-membered aromatic monocyclic or bicyclic group containing from 1 to 4 heteroatoms selected from O, S or N.

As examples of heteroaryl groups, mention may be made of imidazolyl, thiazolyl, oxazolyl, furanyl, thiophenyl, pyrazolyl, oxadiazolyl, tetrazolyl, pyridinyl, pyrazinyl, pyrimidinyl, pyridazinyl, indolyl, benzofuranyl, benzothiophenyl, benzoxazolyl, benzimidazolyl, indazolyl, benzothiazolyl, isobenzothiazolyl, benzotriazolyl, quinolinyl and isoquinolinyl groups.

By way of a heteroaryl comprising 5 to 6 atoms, including 1 to 4 nitrogen atoms, mention may in particular be made of the following representative groups: pyrrolyl, pyrazolyl, 1,2,3-triazolyl, 1,2,4-triazolyl, tetrazolyl and 1,2,3-triazinyl.

Mention may also be made, by way of heteroaryl, of thiophenyl, oxazolyl, furazanyl, 1,2,4-thiadiazolyl, naphthyridinyl, quinoxalinyl, phthalazinyl, imidazo[1,2-a]pyridine, imidazo[2,1-b]thiazolyl, cinnolinyl, benzofurazanyl, azaindolyl, benzimidazolyl, benzothiophenyl, thienopyridyl, thienopyrimidinyl, pyrrolopyridyl, imidazopyridyl, benzoazaindole, 1,2,4-triazinyl, indolizinyl, isoxazolyl, isoquinolinyl, isothiazolyl, purinyl, quinazolinyl, quinolinyl, isoquinolyl, 1,3,4-thiadiazolyl, thiazolyl, isothiazolyl, carbazolyl, and also the corresponding groups resulting from their fusion or from fusion with the phenyl nucleus.

According to an embodiment, the compounds of the invention are compounds having the formula (1) as defined above wherein R¹ is H or F.

According to an embodiment, the compounds of the invention are compounds having the formula (1) as defined above wherein R² is selected from the group consisting of: pyridinyl other than

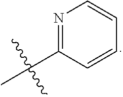 [SMILES: *c1ccccn1]

pyrimidinyl other than

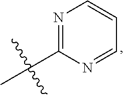 [SMILES: *c1ncccn1]

quinolinyl, thiophenyl, and furanyl groups, said groups being optionally substituted with at least one substituent selected from the group consisting of: halogen, amino, halo(C₁-C₆)alkyl, (C₁-C₆)alkoxy, —C≡C—, and CN.

According to an embodiment, the compounds of the invention are compounds having the formula (1) as defined above wherein R² is selected from the group consisting of: pyridinyl, pyrimidinyl, quinolinyl, thiophenyl, and furanyl groups, said groups being optionally substituted with at least one substituent selected from the group consisting of: halogen, amino, halo(C₁-C₆)alkyl, (C₁-C₆)alkoxy, —C≡C—, and CN, wherein said pyridinyl has the following formula

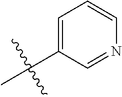 [SMILES: *c1cccnc1]

and wherein said pyrimidinyl has the following formula:

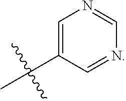 [SMILES: *c1cncnc1]

According to an embodiment, the compounds of the invention are compounds having the formula (1) as defined above wherein R¹ is H or F, and R² is selected from the group consisting of: pyridinyl other than

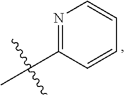 [SMILES: *c1ccccn1]

pyrimidinyl other than

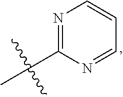 [SMILES: *c1ncccn1]

quinolinyl, thiophenyl, and furanyl groups, said groups being optionally substituted with at least one substituent selected from the group consisting of: halogen, amino, halo(C₁-C₆)alkyl, (C₁-C₆)alkoxy, —C≡C—, and CN.

As preferred compounds according to the invention, the followings may be mentioned:

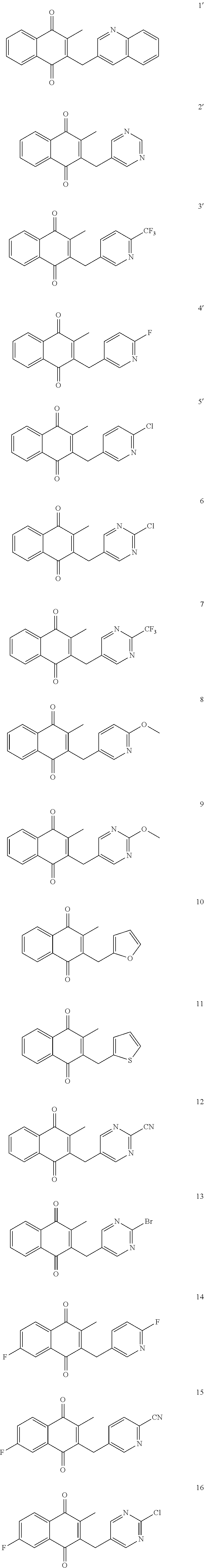

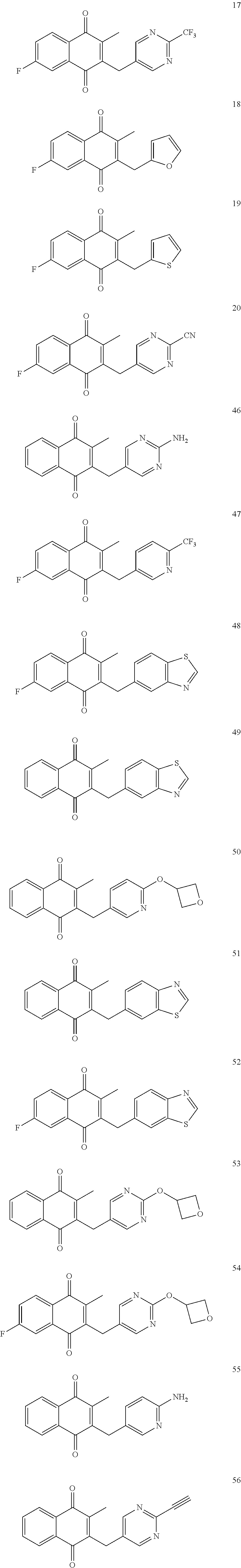

The compounds of formula (1) according to the invention are for example used as drug-candidates or compounds with therapeutic properties, or as ligands or components for active oxygen barriers in plastic polymers for food and drink preservation.

The present invention also relates to a process for the preparation of a compound having the formula (1):

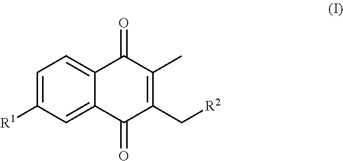

wherein:
R¹ is selected from the group consisting of: H, F, (C₁-C₆)alkoxy, and halo(C₁-C₆)alkyl; and
R² is a heteroaryl group as defined above, said heteroaryl group being optionally substituted with at least one substituent selected from the group consisting of:
   halogen,
   halo(C₁-C₆)alkyl,
   (C₁-C₆)alkoxy,
   CN,
   (C₁-C₆)alkyl,
   NRₐR_{b}, Rₐ and R_{b}, identical or different, being independently H or a (C₁-C₆)alkyl, such as NH₂,
   (C₂-C₆)alkynyl, such as —C≡C—,
   OR_{c}, R_{c} being a cycloheteroalkyl, preferably an oxetanyl group,
   NO₂,
   SF₃,
   SF₅,
   —C(═O)—(C₁-C₆)alkyl,
   halo(C₁-C₆)alkoxy,
   (C₁-C₆)alkoxy,

said process comprising the preparation of a compound having the following formula (IV):

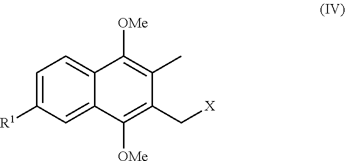

wherein:
R¹ is selected from the group consisting of: H, F, (C₁-C₆)alkoxy, and halo(C₁-C₆)alkyl; and
X is Cl or Br,

by the chloromethylation or bromomethylation of a compound having the following formula (V):

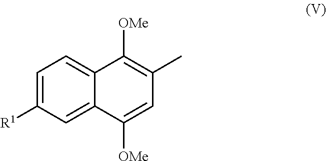

wherein R¹ is selected from the group consisting of: H, F, (C₁-C₆)alkoxy, and halo(C₁-C₆)alkyl.

Depending on the nature of the R₂ group, the compounds of formula (I) may be further submitted to a post-functionalization step, especially for modifying the nature of the possible substituent present on the heteroaryl group.

According to an embodiment, the chloromethylation or bromomethylation step as defined above is carried out with a mixture of hydrochloric acid or hydrobromic acid with paraformaldehyde (or aqueous formaldehyde) in the presence of a solvent selected from the group consisting of: water, acetic acid, and dioxane.

Preferably, the chloromethylation or bromomethylation step is carried out in a mixture of hydrochloric acid or hydrobromic acid and paraformaldehyde.

According to an embodiment, the chloromethylation or bromomethylation step is carried out for a duration comprised from one hour to 24 hours, preferably for 3 hours.

According to an embodiment, the chloromethylation or bromomethylation step is carried out at a temperature comprised from 20° C. to 100° C., preferably at 80° C.

Preferably, the chloromethylation or bromomethylation step mentioned above is a chloromethylation step. Therefore, preferably, the obtained compounds from the compounds of formula (V) as defined above are compounds having the following formula (IV-1):

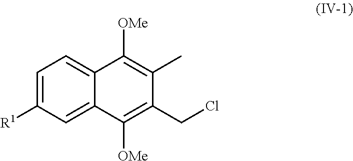

R¹ being as defined above in formula (IV).

Preferably, the chloromethylation step is carried out in a mixture of hydrochloric acid and paraformaldehyde.

According to an embodiment, the chloromethylation step is carried out for a duration comprised from one hour to 24 hours, preferably for 3 hours when R¹ is H, or 18 hours R¹ is F.

According to an embodiment, the chloromethylation step is carried out at a temperature comprised from 20° C. to 100° C., preferably at 80° C. when R¹ is H, or 60° C. when R¹ is F.

According to an embodiment, the process of the invention further comprises the pallado-catalyzed Suzuki coupling of the compound of formula (IV) as defined above with a boronic acid compound having the formula (III) or (III′):

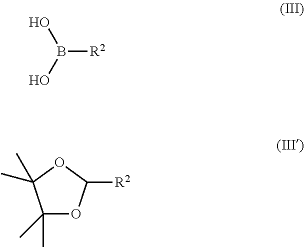

wherein R² is as defined above in formula (I), in order to obtain a compound having the following formula (II):

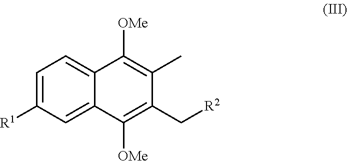

R¹ and R² being as defined above in formula (1).

According to an embodiment, the pallado-catalyzed coupling is carried out with a palladium catalyst selected from the group consisting of: Pd(PPh₃)₄, PdCl₂, PdCl₂(dppf), Pd(OAc)₂ and PPh₃, and with a base selected from the group consisting of: Na₂CO₃, K₂CO₃, KOtBu, Cs₂CO₃, NaOH, and NEt₃, or with K₃PO₄ in toluene.

Preferably, the pallado-catalyzed coupling is carried out with a palladium catalyst being Pd(PPh₃)₄, and with a base being Na₂CO₃.

Preferably, the amount of palladium catalyst is comprised from 2 to 5% in moles in comparison with the number of moles of compound of formula (IV) or (IV-1).

Preferably, the amount of base is comprised from 2 to 5 equivalents, and is preferably 2.1 equivalents.

According to an embodiment, the pallado-catalyzed coupling is carried out in the presence of a solvent selected from the group consisting of: dimethoxyethane (DME)/water, tetrahydrofuran (THF)/water, dioxane/water, dimethylformamide (DMF)/water, toluene/water, preferably in a mixture DME/water.

According to an embodiment, the pallado-catalyzed coupling is carried out for a duration comprised from 30 minutes to 6 hours, preferably for 1 hour.

According to an embodiment, the pallado-catalyzed coupling is carried out at a temperature comprised from 70° C. to 150° C., preferably at 100° C.

According to an embodiment, the process of the invention further comprises an oxidative demethylation step of the compound of formula (II) as defined above in the presence of an oxidant, in order to obtain the compound of formula (1) as defined above.

Preferably, the oxidant is selected from the group consisting of: ceric ammonium nitrate (CAN), silver oxide (AgO/Ag₂O), OsO₄/NalO₄, oxone, BBr₃ with O₂ or open air, and boron trichloride/Tetra-n-butylammonium iodide (BCl₃/TBAI) with O₂ or open air.

According to an embodiment, the oxidative demethylation step is carried out in the presence of a solvent selected from the group consisting of: a mixture of acetonitrile and water, dichloromethane for BBr₃ or BCl₃/TBAI, preferably in a mixture of acetonitrile and water.

According to an embodiment, the oxidative demethylation step is carried out for a duration comprised from 10 minutes to 6 hours, preferably for 1 hour.

According to an embodiment, the oxidative demethylation step is carried out at a temperature comprised from 4° C. to 50° C., preferably at room temperature (22-25° C.).

According to an embodiment of the process according to the invention, the compound of formula (V) is prepared by reacting a compound having the following formula (VI):

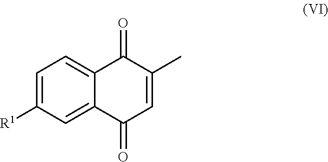

wherein R¹ is selected from the group consisting of: H, F, (C₁-C₆)alkoxy, and halo(C₁-C₆)alkyl,
with a reducing agent,
followed by a methylation step, in order to obtain the compound of formula (V) as defined above.

The compound of formula (V) as defined above is thus obtained from the compound of formula (VI) by the implementation of a reduction step, followed by a methylation step.

According to an embodiment, the reducing agent is selected from the group consisting of: SnCl₂ and HCl, and sodium dithionite.

According to an embodiment, the reduction step is carried out in the presence of a solvent selected from the group consisting of: MeOH, EtOH, or MeOH/water, EtOH/water for sodium dithionite, preferably in ethanol or methanol.

According to an embodiment, the reduction step is carried out for a duration comprised from one hour to 4 hours, preferably for 2 hours.

According to an embodiment, the reduction step is carried out at a temperature comprised from 15° C. to 50° C., preferably at room temperature (22-25° C.).

According to an embodiment, the methylation step is carried out with a methylation agent, said methylation agent being selected from the group consisting of: Me₂SO₄, and ICH₃.

According to an embodiment, the methylation step is carried out in the presence of a solvent being acetone.

According to an embodiment, the methylation step is carried out in the presence of a base selected from the group consisting of NaOH, and KOH.

According to an embodiment, the methylation step is carried out for a duration comprised from one hour to 16 hours, preferably for 4 hours.

According to an embodiment, the methylation step is carried out at a temperature comprised from 40° C. to 60° C., preferably at 60° C.

According to an embodiment, R² is selected from the group consisting of: pyridinyl, pyrimidinyl, quinolinyl, thiophenyl, and furanyl groups, said groups being optionally substituted with at least one substituent selected from the group consisting of: halogen, halo(C₁-C₆)alkyl, (C₁-C₆)alkoxy, and CN.

According to an embodiment, R¹ is H or F.

The present invention also relates to a compound having the following formula (II):

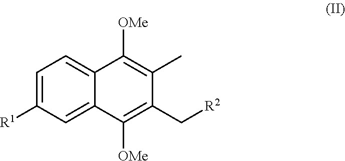

wherein:
R¹ is selected from the group consisting of: H, F, (C₁-C₆)alkoxy, and halo(C₁-C₆)alkyl; and
R² is a heteroaryl group as defined above in formula (1), said heteroaryl group being optionally substituted with at least one substituent selected from the group consisting of:
   halogen,
   halo(C₁-C₆)alkyl,
   (C₁-C₆)alkoxy,
   CN,
   (C₁-C₆)alkyl,
   NRₐR_{b}, Rₐ and R_{b}, identical or different, being independently H or a (C₁-C₆)alkyl, such as NH₂,
   (C₂-C₆)alkynyl, such as —C≡C—,
   OR_{c}, R_{c} being a cycloheteroalkyl, preferably an oxetanyl group,
   NO₂,
   SF₃,
   SF₅,
—C(═O)—(C₁-C₆)alkyl,
   halo(C₁-C₆)alkoxy,
   (C₁-C₆)alkoxy, provided that the compound of formula (II) is different from the following compound:

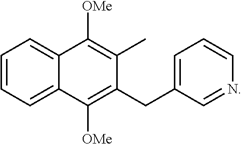 [SMILES: COc1c(C)c(Cc2cccnc2)c(OC)c2ccccc12]

As compounds of formula (II), the followings may be mentioned:

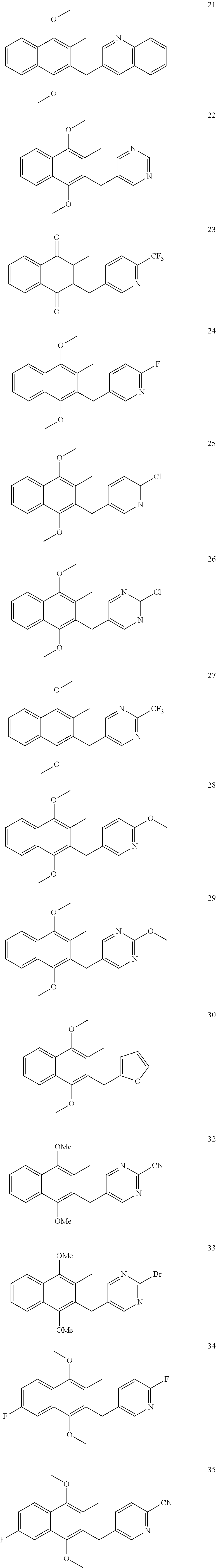

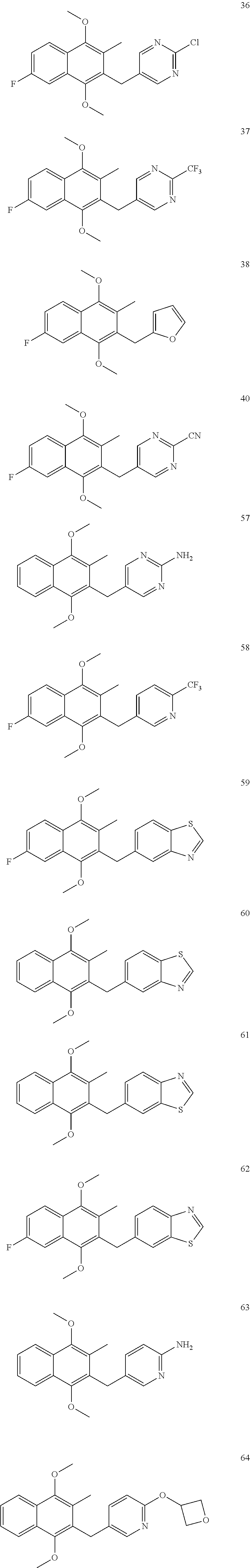

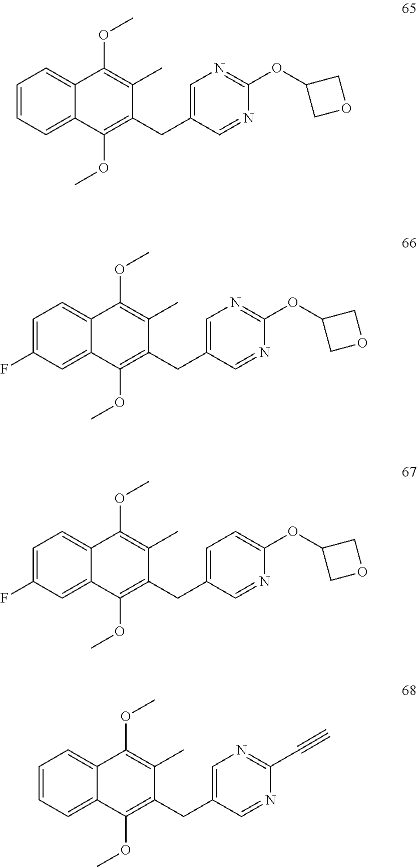 [SMILES: COc1c(C)c(Cc2cnc(OC3COC3)nc2)c(OC)c2ccccc12 ; COc1c(C)c(Cc2cnc(OC3COC3)nc2)c(OC)c2cc(F)ccc12 ; COc1c(C)c(Cc2ccc(OC3COC3)nc2)c(OC)c2cc(F)ccc12 ; C#Cc1ncc(Cc2c(C)c(OC)c3ccccc3c2OC)cn1]

The present invention also relates to a compound having the following formula

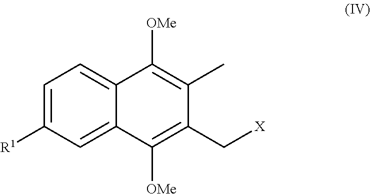

wherein R¹ is selected from the group consisting of: H, F, (C₁-C₆)alkoxy, and halo(C₁-C₆)alkyl, provided that the compound of formula (IV) is different from the following compounds:

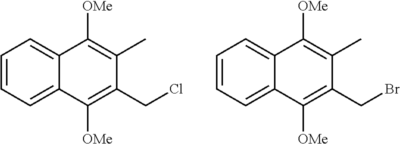

As compounds of formula (IV), the followings may be mentioned:

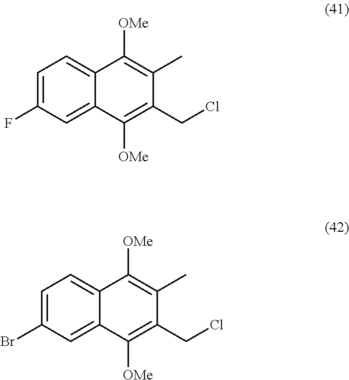 [SMILES: COc1c(C)c(CCl)c(OC)c2cc(F)ccc12 ; COc1c(C)c(CCl)c(OC)c2cc(Br)ccc12]

The present invention also relates to a compound having the following formula

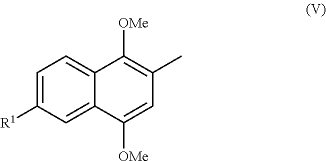

wherein R¹ is selected from the group consisting of: F, (C₁-C₆)alkoxy, and halo(C₁-C₆)alkyl.

As compounds of formula (V), the followings may be mentioned:

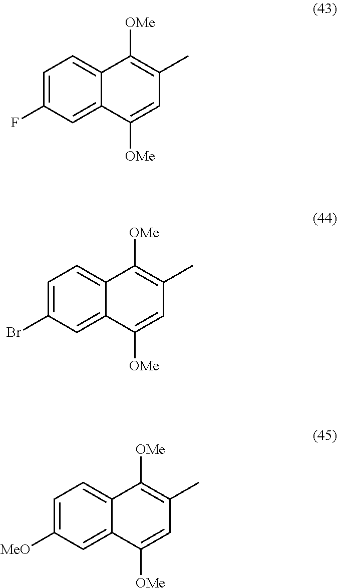 [SMILES: COc1cc(C)c(OC)c2ccc(F)cc12 ; COc1cc(C)c(OC)c2ccc(Br)cc12 ; COc1ccc2c(OC)c(C)cc(OC)c2c1]

### EXAMPLES

Heteroarylboronic acids and reactants were purchased from commercial sources, such as Fluorochem, Sigma-Aldrich and Alfa Aesar. 1,4-dimethoxy-2-methylnaphthalene and 6-fluoro-1,4-dimethoxy-2-methylnaphthalene were synthetized according to a previously published method (T. Mller, L. Johann, B. Jannack, M. Bruckner, D. A. Lanfranchi, H. Bauer, C. Sanchez, V. Yardleyll, C. Deregnaucourt, J. Schrevel, M. Lanzer, R. H. Schirmer, E. Davioud-Charvet, J. Am. Chem. Soc. 2011, 133, 30, 11557-11571). The 6-fluoro-1,4-dimethoxy-2-methylnaphthalene was synthesized from the 6-fluoro-menadione, prepared according to Cesar Rodo E., Feng L., Jida, M., Ehrhardt K., Bielitza M., Boilevin J., Lanzer M., Williams D. L., Lanfranchi, D. A., Davioud-Charvet, E. A platform of regioselective methodologies to access to polysubstituted 2-methyl-1,4-naphthoquinones derivatives: scope and limitations. Eur. J. Org. Chem. 2016, 11, 1982-1993. doi: 10.1002/ejoc.201600144.

The compounds of formula (1) according to the invention are prepared according to the following general scheme:

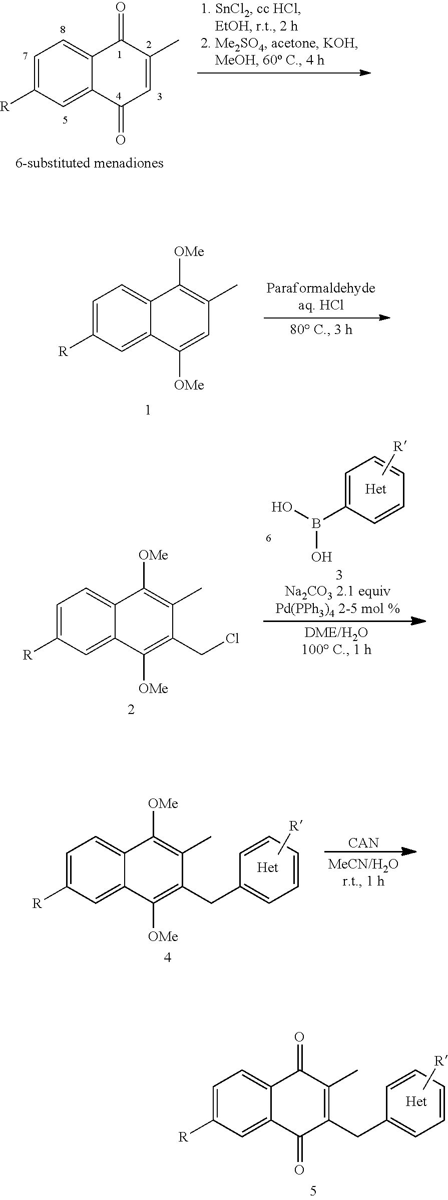

### Preparation of Compounds of Formula (II) (by Suzuki Reaction Between 2-Methyl-3-Chloromethyl-1,4,-Dimethoxynaphtalene and Boronic Acids of Formula (III))

The compounds of formula (II) wherein R¹═H are prepared according to the following reaction scheme:

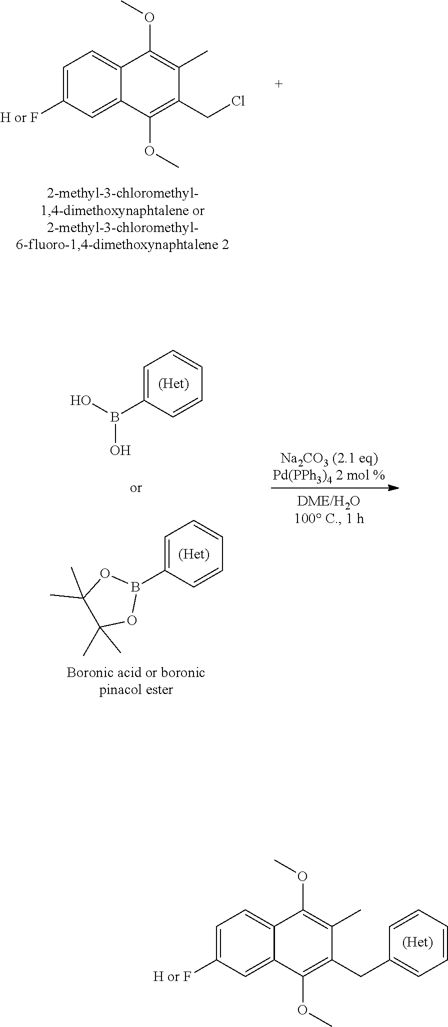

### Synthesis of 2-(Chloromethyl)-1,4-dimethoxy-3-methylnaphthalene

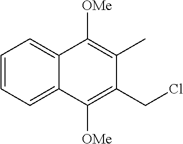 [SMILES: COc1c(C)c(CCl)c(OC)c2ccccc12]

1,4-dimethoxy-2-methylnaphthalene (1 equiv., 4 g, 19.8 mmol), paraformaldehyde (5 equiv., 3.13 g, 2.89 mL, 98.9 mmol) and 37% C, aqueous HCl (50 mL) were heated at 80° C. during 2 h. The OMe mixture was cooled down, diluted with water and extracted three times with EtOAc. The reunited organic layers were washed with brine, dried over MgSO₄ and the solvent was removed under reduced pressure. The crude oil was purified by silica gel chromatography using 8:2 Cyclohexane:Toluene as eluant system to afford 4.086 g (81% yield) of 2-(chloromethyl)-1,4-dimethoxy-3-methylnaphthalene as a colorless oil which crystallized on standing. ¹H NMR (400 MHz, CDCl₃) δ 8.17-8.00 (m, 2H), 7.64-7.41 (m, 2H), 4.92 (s, 2H), 4.04 (s, 3H), 3.89 (s, 3H), 2.54 (s, 3H).

### General procedure for the Suzuki coupling between 2-(chloromethyl)-1,4-dimethoxy-3-methylnaphthalene and heteroarylboronic acid

In a sealable tube, 2-(chloromethyl)-1,4-dimethoxy-3-methylnaphthalene (1 equiv), the corresponding heteroarylboronic acid (1.2 equiv) and sodium carbonate (2.1 equiv) were dissolved in a 2:1 mixture of dimethoxyethane:water (0.15M). The mixture was bubbled 30 min with argon, and then tetrakis(triphenylphosphine)palladium (2-5 mol %) was added at once. The tube was sealed and the mixture was heated 1 h at 100° C. under stirring. The mixture was then, allowed to cool down to room temperature, diluted with water and extracted three times with ethyl acetate. Reunited organic layers were washed with brine, dried over magnesium sulfate and the solvent was removed under reduced pressure to afford a crude, which was purified on silica gel chromatography using the adequate eluant system to afford the corresponding coupling product.

| Boronic acid of formula (III) | Compound of formula (II) | Yield |
|---|---|---|
| 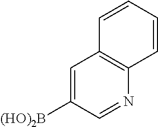 [SMILES: OB(O)c1cnc2ccccc2c1] | 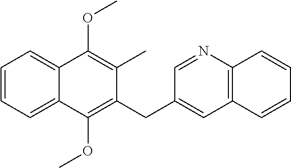 [SMILES: COc1c(C)c(Cc2cnc3ccccc3c2)c(OC)c2ccccc12] | 97% |
| 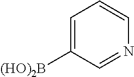 | 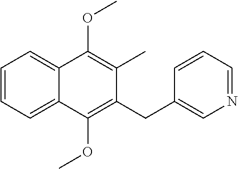 [SMILES: COc1c(C)c(Cc2cccnc2)c(OC)c2ccccc12] | 71% |
| 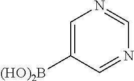 | 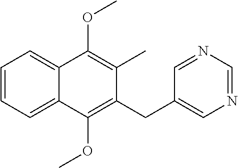 [SMILES: COc1c(C)c(Cc2cncnc2)c(OC)c2ccccc12] | 87% |
| 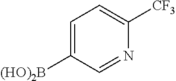 [SMILES: OB(O)c1ccc(C(F)(F)F)nc1] | 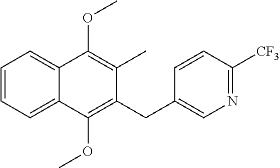 [SMILES: COc1c(C)c(Cc2ccc(C(F)(F)F)nc2)c(OC)c2ccccc12] | 97% |
| 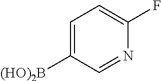 | 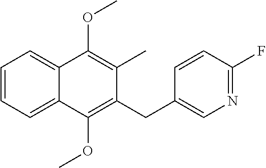 [SMILES: COc1c(C)c(Cc2ccc(F)nc2)c(OC)c2ccccc12] | 85% |
| 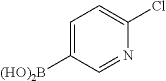 | 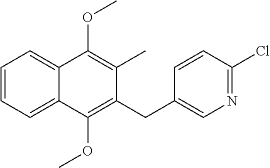 [SMILES: COc1c(C)c(Cc2ccc(Cl)nc2)c(OC)c2ccccc12] | 87% |
| 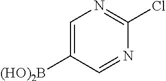 | 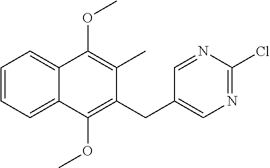 [SMILES: COc1c(C)c(Cc2cnc(Cl)nc2)c(OC)c2ccccc12] | 97% |
| 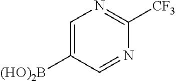 [SMILES: OB(O)c1cnc(C(F)(F)F)nc1] | 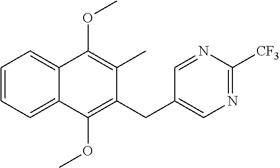 [SMILES: COc1c(C)c(Cc2cnc(C(F)(F)F)nc2)c(OC)c2ccccc12] | 71% |
| 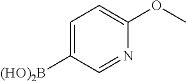 | 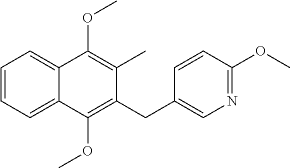 [SMILES: COc1ccc(Cc2c(C)c(OC)c3ccccc3c2OC)cn1] | 69% |
| 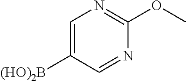 | 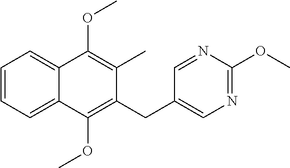 [SMILES: COc1ncc(Cc2c(C)c(OC)c3ccccc3c2OC)cn1] | 75% |
| 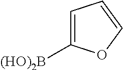 | 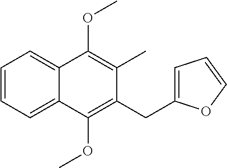 [SMILES: COc1c(C)c(Cc2ccco2)c(OC)c2ccccc12] | 100% |

### Preparation of 3-((1,4-dimethoxy-3-methylnaphthalen-2-yl)methyl) quinoline (21)

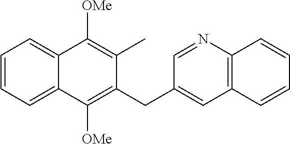 [SMILES: COc1c(C)c(Cc2cnc3ccccc3c2)c(OC)c2ccccc12]

5 mol % Pd(PPh₃), Eluant Cyclohexane:Ethyl acetate (7:3), yellowish solid, 97% yield. ¹H NMR (500 MHz, CDCl₃) δ 8.92 (d, J=2.3 Hz, 1H), 8.17-8.10 (m, 2H), 8.07 (dd, J=8.4, 1.0 Hz, 1H), 7.69-7.59 (m, 3H), 7.58-7.50 (m, 2H), 7.46 (ddd, J=8.1, 6.7, 1.2 Hz, 1H), 4.44 (s, 2H), 3.87 (s, 6H), 2.29 (s, 3H). ¹³C NMR (126 MHz, CDCl₃) δ 151.95, 150.90, 150.77, 146.96, 133.87, 133.49, 129.24, 128.85, 128.36, 128.30, 127.90, 127.58, 127.38, 126.73, 126.19, 125.81, 122.69, 122.46, 62.58, 61.61, 30.50, 12.89. HRMS (ESI) calcd. for C₂₃H₂₂NO₂: 344.1645. Found: 344.1659 (MH⁺).

### Preparation of 3-((1,4-dimethoxy-3-methylnaphthalen-2-yl)methyl) pyridine (21′)

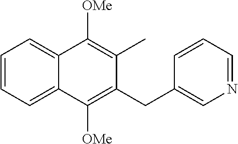 [SMILES: COc1c(C)c(Cc2cccnc2)c(OC)c2ccccc12]

5 mol % Pd(PPh₃), Eluant Cyclohexane:Ethyl acetate (1:1), yellowish solid, 71% yield. ¹H NMR (500 MHz, CDCl₃) δ 8.50N (dd, J=2.3, 1.0 Hz, 1H), 8.41 (dd, J=4.8, 1.6 Hz, 1H), 8.12-8.04 (m, 2H), 7.63-7.42 (m, 2H), 7.39-7.32 (m, 1H), 7.13 (ddd, J=7.8, 4.8, 0.9 Hz, 1H), 4.25 (s, 2H), 3.86 (s, 3H), 3.85 (s, 3H), 2.26 (s, 3H). ¹³C NMR (126 MHz, CDCl₃) δ 150.75, 150.70, 149.92, 147.50, 136.08, 135.67, 128.28, 128.00, 127.30, 126.64, 126.12, 125.77, 123.55, 122.60, 122.43, 62.45, 61.58, 30.28, 12.83. HRMS (ESI) calcd. for C₁₉H₂₀NO₂: 294.1489. Found: 294.1490 (MH⁺).

### Preparation of 5-((1,4-dimethoxy-3-methylnaphthalen-2-yl)methyl) pyrimidine (22)

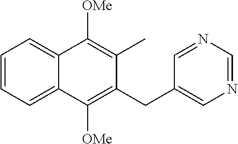 [SMILES: COc1c(C)c(Cc2cncnc2)c(OC)c2ccccc12]

5 mol % Pd(PPh₃), Eluant Cyclohexane:Ethyl acetate (1:1), N beige solid, 87% yield. ¹H NMR (500 MHz, CDCl₃) δ 9.04 (s, IN 1H), 8.54 (s, 2H), 8.13-7.98 (m, 2H), 7.61-7.41 (m, 2H), 4.22 (s, 2H), 3.88 (s, 3H), 3.86 (s, 3H), 2.29 (s, 3H). ¹³C NMR (126 MHz, CDCl₃) δ 156.91, 156.77, 150.91, 150.80, 133.90, 128.49, 127.29, 126.68, 126.40, 125.99, 122.62, 122.52, 62.46, 61.66, 28.10, 12.92. HRMS (ESI) calcd. for C₁₈H₁₉N₂O₂: 295.1441. Found: 295.1450 (MH⁺).

### Preparation of 5-((1,4-dimethoxy-3-methylnaphthalen-2-yl)methyl)-2-(trifluoromethyl)pyridine (23)

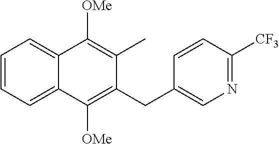 [SMILES: COc1c(C)c(Cc2ccc(C(F)(F)F)nc2)c(OC)c2ccccc12]

5 mol % Pd(PPh₃), Eluant Cyclohexane:Ethyl acetate (9:1), translucid oil, 97% yield. ¹H NMR (500 MHz, CDCl₃) δ 8.64 (d, J=1.4 Hz, 1H), 8.16-8.04 (m, 2H), 7.61-7.46 (m, 4H), 4.31 (s, 2H), 3.87 (s, 3H), 3.86 (s, 3H), 2.26 (s, 3H). ¹³C NMR (101 MHz, CDCl₃) δ 150.81, 150.77, 150.08, 145.99 (q, J=34.6 Hz), 139.66, 136.79, 128.42, 127.24, 127.05, 126.29, 126.17, 125.88, 122.55, 121.78 (q, J=273.7 Hz), 120.31 (q, J=2.7 Hz), 62.33, 61.50, 30.13, 12.77. ¹⁹F NMR (471 MHz, CDCl₃) δ−67.68. HRMS (ESI) calcd. for C₂₀H₁₉F₃NO₂: 362.1362. Found: 362.1368 (MH⁺).

### Preparation of 5-((1,4-dimethoxy-3-methylnaphthalen-2-yl)methyl)-2-fluoropyridine (24)

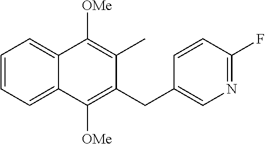 [SMILES: COc1c(C)c(Cc2ccc(F)nc2)c(OC)c2ccccc12]

2 mol % Pd(PPh₃), Eluant Cyclohexane:Ethyl acetate (9:1), white solid, 89% yield. ¹H NMR (400 MHz, CDCl₃) δ 7.94-7.70 (m, 3H), 7.40-7.19 (m, 3H), 6.54 (ddd, J=8.4, 3.1, 0.6 Hz, 1H), 3.98 (s, 1H), 3.63 (s, 3H), 3.61 (s, 3H), 2.03 (s, 3H). ¹⁹F NMR (377 MHz, CDCl₃) δ−72.09 (d, J=8.0 Hz). ¹³C NMR (101 MHz, CDCl₃) δ 162.42 (d, J=237.2 Hz), 150.78, 150.65, 146.98 (d, J=14.4 Hz), 141.02 (d, J=7.6 Hz), 133.65 (d, J=4.5 Hz), 128.33, 127.92, 127.29, 126.39, 126.21, 125.84, 122.59, 122.46, 109.33 (d, J=37.4 Hz), 62.43, 61.58, 29.38 (d, J=1.5 Hz), 12.78. HRMS (ESI) calcd. for C₁₉H₁₉FNO₂: 312.1394. Found: 312.1403 (MH⁺).

### Preparation of 2-chloro-5-((1,4-dimethoxy-3-methylnaphthalen-2-yl)methyl)pyridine (25)

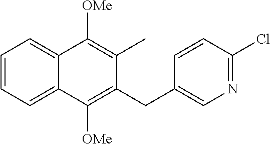 [SMILES: COc1c(C)c(Cc2ccc(Cl)nc2)c(OC)c2ccccc12]

5 mol % Pd(PPh₃), Eluant Cyclohexane:Ethyl acetate (9:1), white solid, 87% yield. ¹H NMR (400 MHz, CDCl₃) δ 8.19 (dd, J=2.6, 0.8 Hz, 1H), 8.07-7.91 (m, 2H), 7.51-7.38 (m, 2H), 7.26 (dd, J=8.2, 2.5 Hz, 1H), 7.08 (dd, J=8.2, 0.7 Hz, 1H), 4.13 (s, 2H), 3.78 (s, 3H), 3.77 (s, 3H), 2.18 (s, 3H). ¹³C NMR (101 MHz, CDCl₃) δ 150.80, 150.72, 149.55, 149.22, 138.71, 135.12, 128.38, 127.56, 127.28, 126.34, 126.27, 125.88, 124.18, 122.60, 122.48, 62.46, 61.60, 29.57, 12.82. HRMS (ESI) calcd. for C₁₉H₁₉ClNO₂: 328.1099. Found: 328.1102 (MH⁺).

### Preparation of 2-chloro-5-((1,4-dimethoxy-3-methylnaphthalen-2-yl)methyl)pyrimidine (26)

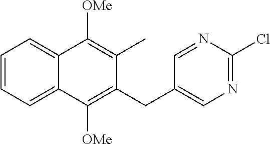 [SMILES: COc1c(C)c(Cc2cnc(Cl)nc2)c(OC)c2ccccc12]

2 mol % Pd(PPh₃), Eluant Cyclohexane:Ethyl acetate (8:2), white solid, 97% yield. ¹H NMR (400 MHz, CDCl₃) δ 8.43 (s, 2H), 8.13-8.00 (m, 2H), 7.61-7.43 (m, 2H), 4.18 (s, 2H), 3.88 (s, 3H), 3.85 (s, 3H), 2.29 (s, 3H). ¹³C NMR (101 MHz, CDCl₃) δ 159.40, 150.96, 150.73, 132.58, 128.54, 127.23, 126.50, 126.22, 126.05, 125.67, 122.59, 122.52, 62.42, 61.63, 27.32, 12.90. HRMS (ESI) calcd. for C₁₈H₁₈ClN₂O₂: 329.1051. Found: 329.1054 (MH⁺).

### Preparation of 5-((1,4-dimethoxy-3-methylnaphthalen-2-yl)methyl)-2-(trifluoromethyl)pyrimidine (27)

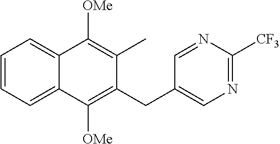 [SMILES: COc1c(C)c(Cc2cnc(C(F)(F)F)nc2)c(OC)c2ccccc12]

2 mol % Pd(PPh₃), Eluant Cyclohexane:Ethyl acetate (8:2), white solid, 71% yield. ¹H NMR (400 MHz, CDCl₃) δ 8.70 (s, 2H), 8.23-7.97 (m, 2H), 7.68-7.33 (m, 2H), 4.28 (s, 2H), 3.90 (s, 3H), 3.86 (s, 3H), 2.30 (s, 3H). ¹⁹F NMR (377 MHz, CDCl₃) δ−70.15. HRMS (ESI) calcd. for C₁₇H₁₂F₃N₂O₂: 333.0845. Found: 333.0868 (MH⁺).

### Preparation of 5-((1,4-dimethoxy-3-methylnaphthalen-2-yl)methyl)-2-methoxypyridine (28)

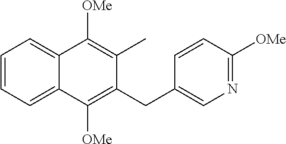 [SMILES: COc1ccc(Cc2c(C)c(OC)c3ccccc3c2OC)cn1]

2 mol % Pd(PPh₃)₄, Eluant Cyclohexane:Ethyl acetate (95:5), translucid oil, 69% yield. ¹H NMR (400 MHz, CDCl₃) δ 8.14-8.04 (m, 2H), 8.02-7.94 (m, 1H), 7.58-7.44 (m, 2H), 7.32 (dd, J=8.5, 2.6 Hz, 1H), 6.61 (dd, J=8.6, 0.7 Hz, 1H), 4.16 (s, 2H), 3.89 (s, 3H), 3.85 (s, 3H), 3.85 (s, 3H), 2.28 (s, 3H). ¹³C NMR (101 MHz, CDCl₃) δ 162.80, 150.63, 150.59, 146.04, 138.87, 128.68, 128.57, 128.16, 127.33, 126.73, 125.98, 125.67, 122.57, 122.39, 110.72, 62.43, 61.55, 53.41, 29.37, 12.75. HRMS (ESI) calcd. for C₂₀H₂₂NO₃: 324.1594. Found: 324.1617 (MH⁺).

### Preparation of 5-((1,4-dimethoxy-3-methylnaphthalen-2-yl)methyl)-2-(methoxy)pyrimidine (29)

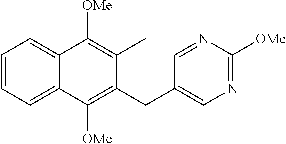 [SMILES: COc1ncc(Cc2c(C)c(OC)c3ccccc3c2OC)cn1]

2 mol % Pd(PPh₃)₄, Eluant Cyclohexane:Ethyl acetate (60:40), translucid oil, 71% yield. ¹H NMR (400 MHz, CDCl₃) δ 8.31 (s, 2H), 8.18-7.95 (m, 2H), 7.60-7.44 (m, 2H), 4.13 (s, 2H), 3.95 (s, 3H), 3.88 (s, 3H), 3.85 (s, 3H), 2.30 (s, 3H). HRMS (ESI) calcd. for C₁₉H₂₀N₂NaO₃: 347.1366. Found: 347.1357 (MNa⁺).

### Preparation of 2-((1,4-dimethoxy-3-methylnaphthalen-2-yl)methyl)furan (30)

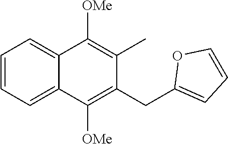 [SMILES: COc1c(C)c(Cc2ccco2)c(OC)c2ccccc12]

2 mol % Pd(PPh₃)₄, Eluant Cyclohexane:Ethyl acetate (99:1 up to 98:2), translucid oil, 99% yield. ¹H NMR (400 MHz, CDCl₃) δ 8.19-8.03 (m, 2H), 7.62-7.46 (m, 2H), 7.34 (dd, J=1.8, 0.9 Hz, 1H), 6.27 (dd, J=3.2, 1.9 Hz, 1H), 5.85 (dq, J=3.2, 1.1 Hz, 1H), 4.27 (s, 2H), 3.92 (s, 3H), 3.90 (s, 3H), 2.41 (s, 3H). ¹³C NMR (101 MHz, CDCl₃) δ 154.30, 150.68, 150.37, 141.15, 128.18, 127.29, 127.06, 126.87, 125.98, 125.54, 122.62, 122.31, 110.37, 105.90, 62.63, 61.46, 26.34, 12.45. HRMS (ESI) calcd. for C₁₈H₁₉O₃: 283.1329. Found: 283.1334 (MH⁺).

### Post-Modifications of Suzuki Coupling Products

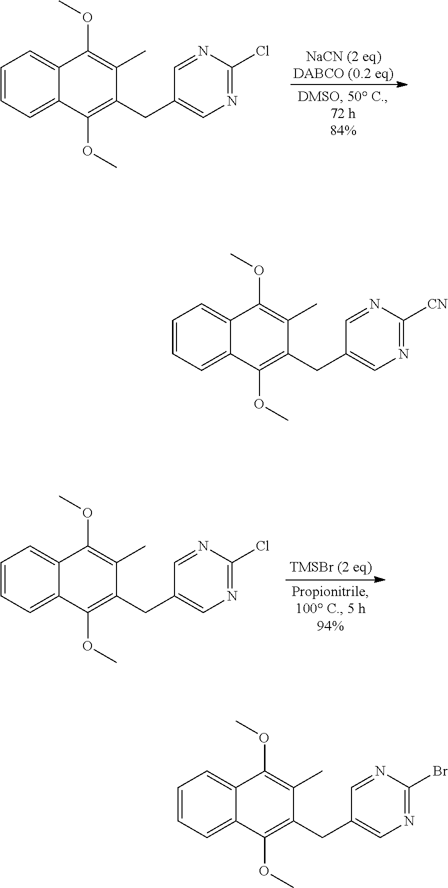 [SMILES: COc1c(C)c(Cc2cnc(Cl)nc2)c(OC)c2ccccc12 ; COc1c(C)c(Cc2cnc(C#N)nc2)c(OC)c2ccccc12 ; COc1c(C)c(Cc2cnc(Cl)nc2)c(OC)c2ccccc12 ; COc1c(C)c(Cc2cnc(Br)nc2)c(OC)c2ccccc12]

### Preparation of 5-((1,4-dimeth oxy-3-methylnaphthalen-2-yl)methyl) pyrimidine-2-carbonitrile (32)

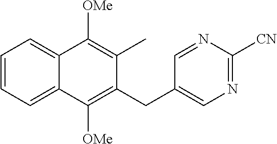 [SMILES: COc1c(C)c(Cc2cnc(C#N)nc2)c(OC)c2ccccc12]

To a solution of sodium cyanide (2 equiv., 44.7 mg, 0.912 mmol) and DABCO (0.2 equiv., 10.8 mg, 0.0912 mmol), in a mixture of DMSO (1.5 mL) and water (0.2 mL), 2-chloro-5-((1,4-dimethoxy-3-methylnaphthalen-2-yl)methyl)pyrimidine (1 eq., 150 mg, 0.456 mmol) in DMSO (1.5 mL) was added dropwise and the reaction mixture was stirred at 50° C. 20 h. After TLC analysis showed complete conversion, the mixture was allowed to cool down to room temperature and was extracted twice with diethyl ether. The reunited organic layers were washed with brine, dried over MgSO₄ and the solvent was removed under reduced pressure. The product was purified by silica gel chromatography was performed using 75:25 Cyclohexane:Ethyl Acetate as eluant system to afford 122 mg (84% yield) of 5-((1,4-dimethoxy-3-methylnaphthalen-2-yl)methyl)pyrimidine-2-carbonitrile as white solid. ¹H NMR (400 MHz, CDCl₃) δ 8.64 (s, 2H), 8.16-7.97 (m, 2H), 7.63-7.43 (m, 2H), 4.27 (s, 2H), 3.90 (s, 3H), 3.86 (s, 3H), 2.29 (s, 3H). ¹³C NMR (101 MHz, CDCl₃) δ 157.79, 151.07, 150.80, 143.08, 137.17, 128.69, 127.21, 126.68, 126.19, 125.51, 125.46, 122.60, 122.58, 115.87, 62.40, 61.68, 28.33, 12.98. HRMS (ESI) calcd. for C₁₇H₁₂N₃O₂: 290.0924. Found: 290.0918 (MH⁺).

### Preparation of 2-bromo-5-((1,4-dimethoxy-3-methylnaphthalen-2-yl)methyl)pyrimidine (33)

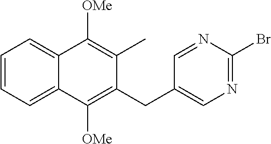 [SMILES: COc1c(C)c(Cc2cnc(Br)nc2)c(OC)c2ccccc12]

To a solution of 2-chloro-5-((1,4-dimethoxy-3-methylnaphthalen-2-yl)methyl)pyrimidine (1 equiv., 150 mg, 0.456 mmol) in propionitrile (1.5 mL) was added trimethylbromosilane (2 equiv., 139 mg, 0.12 mL, 0.912 mmol). A white precipitate appeared. The mixture was heated at reflux 5 h and allowed to cool down to room temperature. The mixture was treated with an aqueous saturated sodium bicarbonate solution. The aqueous layer was extracted three times with ethyl acetate, the reunited organic layers were washed with brine, dried over MgSO4 and the solvent was removed under reduced pressure. The product was purified by silica gel chromatography was performed using 95:5 Toluene:Ethyl Acetate as eluant system to afford 160 mg (94% yield) of 2-bromo-5-((1,4-dimethoxy-3-methylnaphthalen-2-yl)methyl)pyrimidine as a translucid solid. m.p: 99-100° C. ¹H NMR (500 MHz, CDCl₃) δ 8.37 (s, 2H), 8.11-7.98 (m, 2H), 7.65-7.47 (m, 2H), 4.16 (s, 2H), 3.88 (s, 3H), 3.85 (s, 3H), 2.29 (s, 3H). ¹³C NMR (126 MHz, CDCl₃) δ 159.33, 150.97, 150.80, 150.74, 133.07, 128.55, 127.23, 126.52, 126.13, 126.07, 125.67, 122.61, 122.54, 62.44, 61.66, 27.39, 12.92. HRMS (ESI) calcd. for C₁₆H₁₂BrN₂O₂: 343.0077. Found: 343.0099 (MH⁺).

### Preparation of 5-((1,4-dimethoxy-3-methylnaphthalen-2-yl)methyl) pyrimidin-2-amine (57)

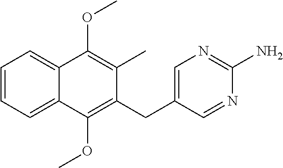 [SMILES: COc1c(C)c(Cc2cnc(N)nc2)c(OC)c2ccccc12]

2 mol % Pd(PPh₃), Eluant Cyclohexane:Ethyl acetate (8/2), white solid, 80% yield. ¹H NMR (400 MHz, CDCl₃) δ 8.04-8.00 (m, 2H), 7.99 (s, 2H), 7.57-7.51 (m, 2H), 6.45 (s, 2H), 3.97 (s, 2H), 3.83 (s, 3H), 3.78 (s, 3H), 2.26 (s, 3H). ¹³C NMR (101 MHz, CDCl₃) δ 162.7, 157.8 (2C), 150.34, 150.30, 129.1, 127.8, 127.1, 126.7, 126.5, 126.3, 122.7, 122.5, 121.5, 62.6, 61.6, 26.8, 12.9. HRMS (ESI+) calcd. for C₁₈H₂₀N₃O₂: 310.1550. Found: 310.1539 (MH⁺). M. p.=198-200° C.

### Preparation of 5-((7-fluoro-1,4-dimethoxy-3-methylnaphthalen-2-yl) methyl)-2-(trifluoromethyl)pyridine (58)

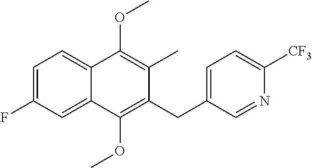 [SMILES: COc1c(C)c(Cc2ccc(C(F)(F)F)nc2)c(OC)c2cc(F)ccc12]

2 mol % Pd(PPh₃), Eluant cyclohexane/ethyl acetate (8/2) then pentane/diethyl ether (gradient from 9/1 to 0/1), colourless oil, 55% yield. ¹H NMR (400 MHz, CDCl₃) δ 8.62 (s, 1H), 8.09 (dd, J=9.2, 5.6 Hz, 1H), 7.66 (dd, J=10.3, 2.5 Hz, 1H), 7.53 (d, J=1.4 Hz, 2H), 7.28 (ddd, J=9.2, 8.3, 2.6 Hz, 1H), 4.30 (s, 2H), 3.84 (s, 6H), 2.24 (s, 3H). ¹³C NMR (101 MHz, CDCl₃) δ 162.4, 159.9, 151.0 (d, ⁵J_{C-F}=1.3 Hz), 150.3 (d, ⁴J_{C-F}=5.4 Hz), 150.1, 146.2 (q, ²J_{C-F}=34.7 Hz), 140.0-138.9 (m), 136.8, 128.6, 128.3 (d, ³J_{C-F}=8.6 Hz), 125.5 (d, ³J_{C-F}=7.2 Hz), 125.4 (d, ³J_{C-F}=9.0 Hz), 121.8 (q, ¹J_{C-F}=273.7 Hz), 120.4 (q, ⁴J_{C-F}=2.7 Hz), 116.6 (d, ²J_{C-F}=25.4 Hz), 106.4 (d, ²J_{C-F}=22.4 Hz), 62.3, 61.7, 30.3, 12.7. ¹⁹F NMR (377 MHz, CDCl₃): δ−67.71, −114.25 (ddd, J=10.1, 8.4, 5.6 Hz). HRMS (ESI+) calcd. for C₂₀H₁₈F₄NO₂: 380.126818. Found: 380.125589 (MH⁺).

### Preparation of 5-((7-fluoro-1,4-dimethoxy-3-methylnaphthalen-2-yl) methyl)benzo[d]thiazole (59)

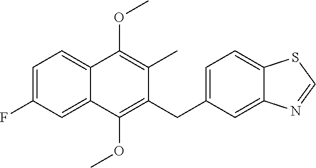 [SMILES: COc1c(C)c(Cc2ccc3scnc3c2)c(OC)c2cc(F)ccc12]

2 mol % Pd(PPh₃), Eluant cyclohexane/ethyl acetate (7/3) then pentane/diethyl ether (7/3), colourless oil, 83% yield. ¹H NMR (400 MHz, CDCl₃) δ 8.97 (s, 1H), 8.10 (dd, J=9.2, 5.6 Hz, 1H), 7.84 (d, J=8.2 Hz, 2H), 7.70 (dd, J=10.4, 2.5 Hz, 1H), 7.34-7.25 (m, 2H), 4.43 (s, 2H), 3.85 (s, 3H), 3.83 (s, 3H), 2.27 (s, 3H). ¹³C NMR (101 MHz, CDCl₃) δ 161.0 (d, ¹J_{C-F}=245.2 Hz), 150.8 (d, ⁵J_{C-F}=1.1 Hz), 150.2 (d, ⁴J_{C-F}=5.3 Hz), 139.0, 130.3, 128.4, 128.3, 126.4 (2C), 126.2 (d, ⁴J_{C-F}=2.4 Hz), 125.23, 125.17 (d, ³J_{C-F}=8.9 Hz), 122.7, 121.7 (2C), 116.1 (d, ²J_{C-F}=25.4 Hz), 106.3 (d, ²J_{C-F}=22.3 Hz), 62.3, 61.6, 32.8, 12.7. ¹⁹F NMR (377 MHz, CDCl₃): δ-114.78 (ddd, J=10.2, 8.5, 5.7 Hz). HRMS (ESI+) calcd. for C₂₁H₁₉FNO₂S: 368.111504. Found: 368.110660 (MH⁺).

### Preparation of 5-((1,4-dimethoxy-3-methylnaphthalen-2-yl)methyl) benzo[d]thiazole (60)

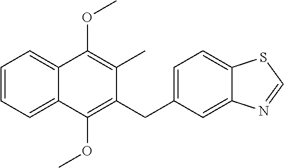 [SMILES: COc1c(C)c(Cc2ccc3scnc3c2)c(OC)c2ccccc12]

2 mol % Pd(PPh₃), Eluant cyclohexane/ethyl acetate (7/3) then pentane/diethyl ether (7/3), white solid, 81% yield. ¹H NMR (400 MHz, CDCl₃) δ 8.94 (s, 1H), 8.22-8.04 (m, 2H), 7.89 (s, 1H), 7.82 (d, J=8.3 Hz, 1H), 7.60-7.48 (m, 2H), 7.31 (d, J=8.2 Hz, 1H), 4.46 (s, 2H), 3.88 (s, 3H), 3.87 (s, 3H), 2.31 (s, 3H). ¹³C NMR (101 MHz, CDCl₃) δ 154.3, 153.8, 150.7, 150.5, 139.2, 131.3, 128.8, 128.1, 127.3, 126.9, 126.3, 125.9, 125.5, 122.7, 122.5, 122.3, 121.6, 62.4, 61.5, 32.7, 12.8. HRMS (ESI+) calcd. for C₂₁H₂₀NO₂S: 350.120926. Found: 350.120240 (MH⁺). M.p.=139-141° C.

### Preparation of 6-((1,4-dimethoxy-3-methylnaphthalen-2-yl)methyl) benzo[d]thiazole (61)

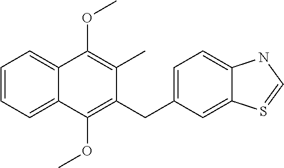

2 mol % Pd(PPh₃), Eluant cyclohexane/ethyl acetate (8/2), colourless oil, 91% yield. ¹H NMR (400 MHz, CDCl₃) δ 8.90 (s, 1H), 8.14-8.10 (m, 2H), 8.03 (d, J=8.4 Hz, 1H), 7.59 (s, 1H), 7.55-7.51 (m, 2H), 7.41-7.37 (m, 1H), 4.43 (s, 2H), 3.87 (s, 3H), 3.86 (s, 3H), 2.28 (s, 3H). ¹³C NMR (101 MHz, CDCl₃) δ 153.5, 151.8, 150.8, 150.7, 138.6, 134.3, 128.9, 128.3, 127.4, 127.2, 127.0, 126.1, 125.7, 123.4, 122.7, 122.4, 120.8, 62.5, 61.6, 32.8, 12.9. HRMS (ESI+) calcd. for C₂₁H₂₀NO₂S: 350.120926. Found: 350.121033 (MH⁺).

### Preparation of 6-((7-fluoro-1,4-dimethoxy-3-methylnaphthalen-2-yl) methyl)benzo[d]thiazole (62)

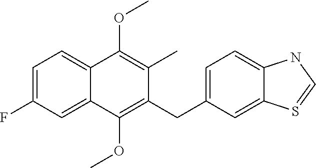

2 mol % Pd(PPh₃), Eluant cyclohexane/ethyl acetate (8/2), colourless oil, 62% yield. ¹H NMR (400 MHz, CDCl₃) δ 8.94 (s, 1H), 8.11 (dd, J=9.2, 5.6 Hz, 1H), 8.04 (br d, J=4.4 Hz, 1H), 7.71 (dd, J=10.4, 2.5 Hz, 1 H), 7.61 (s, 1H), 7.38 (br d, J=2.8 Hz, 1H), 7.28 (td, J=8.8, 2.5 Hz, 1H), 4.41 (s, 2H), 3.85 (s, 3H), 3.84 (s, 3H), 2.26 (s, 3H). ¹³C NMR (101 MHz, CDCl₃) δ 161.0 (d, ¹J_{C-F}=245.4 Hz), 153.8, 152.2, 150.8 (d, ⁴J_{C-F}=1.2 Hz), 150.2 (d, ³J_{C-F}=5.3 Hz), 138.2, 134.7, 130.4, 128.3 (d, ³J_{C-F}=8.6 Hz), 127.1, 126.2 (d, ⁴J_{C-F}=2.5 Hz), 125.3, 125.2, 123.4, 120.7, 116.2 (d, ²J_{C-F}=25.4 Hz), 106.4 (d, ²J_{C-F}=22.3 Hz), 62.3, 61.7, 32.9, 12.7. ¹⁹F NMR (CDC₃, 377 MHz): δ-114.66 (ddd, J=10.2, 8.4, 5.7 Hz). HRMS (ESI+) calcd. for C₂₁H₉NO₂S: 368.111504. Found: 368.111827 (MH⁺).

### Preparation of 5-[(1,4-dimethoxy-3-methylnaphthalen-2-yl)methyl]pyridin-2-amine (63)

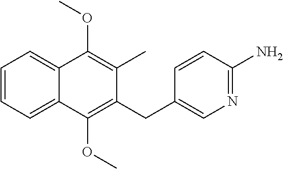 [SMILES: COc1c(C)c(Cc2ccc(N)nc2)c(OC)c2ccccc12]

7 mol % Pd(PPh₃), Eluant ethyl acetate, orange solid, 72% yield. ¹H NMR (400 MHz, CDCl₃) δ 8.08 (tdd, J=5.3, 4.0, 3.2 Hz, 2H), 7.91 (s, 1H), 7.74-7.61 (m, 2H), 7.15 (dd, J=8.5, 2.3 Hz, 1H), 6.38 (d, J=8.4 Hz, 1H), 4.36 (s, 2H), 4.10 (s, 2H), 3.85 (s, 3H), 3.84 (s, 3H), 2.28 (s, 3H). ¹³C {¹H} NMR (101 MHz, CDCl₃) δ 171.2, 156.7, 150.4, 147.1, 137.9, 128.6, 128.5, 125.8, 125.5, 122.4, 122.2, 108.8, 62.3, 61.4, 29.3, 12.6.

### Post-Modifications of Suzuki Coupling Products:

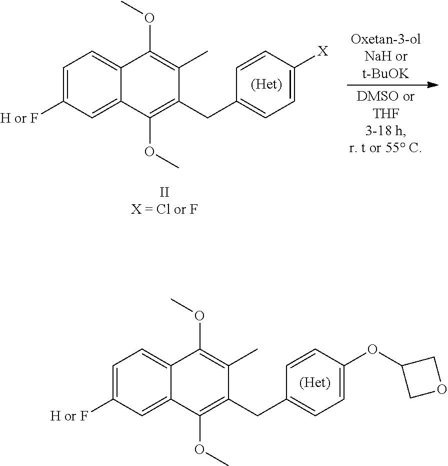

### Preparation of 5-((1,4-dimethoxy-3-methylnaphthalen-2-yl)methyl)-2-(oxetan-3-yloxy)pyridine (64)

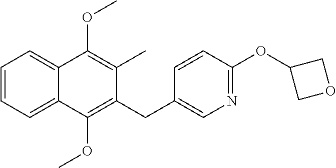 [SMILES: COc1c(C)c(Cc2ccc(OC3COC3)nc2)c(OC)c2ccccc12]

To a solution of oxetan-3-ol (110 μL, 1.73 mmol, 5.0 equiv.) in anhydrous DMSO (800 μL), NaH (42 mg, 1.73 mmol, 5.0 equiv.) was added portion-wise under argon atmosphere. The mixture was stirred until homogenous and was added dropwise to a solution of 5-((1,4-dimethoxy-3-methylnaphthalen-2-yl)methyl)-2-fluoropyridine (108 mg, 0.35 mmol, 1.0 equiv.) in anhydrous DMSO (500 μL), in a sealed tube, and stirred for 18 h at room temperature under argon atmosphere. The reaction mixture was quenched with a 1 M aqueous solution of hydrochloric acid and diluted with dichloromethane. The layers were separated and the aqueous layer was extracted twice with dichloromethane. The organic phase was washed with water, brine, dried over MgSO₄ and concentrated under reduced pressure. The crude residue was purified by flash chromatography on silica gel (Cyclohexane/ethyl acetate, 8/2, v/v, UV) to afford 64 as a colourless oil (64 mg, 50%). ¹H NMR (CDC₃, 400 MHz): δ 8.12-8.04 (m, 2H), 7.88 (d, J=1.8 Hz, 1H), 7.53-7.46 (m, 2H), 7.37 (dd, J=8.5, 2.4 Hz, 1H), 6.66 (d, J=8.5 Hz, 1H), 5.55 (p, J=5.8 Hz, 1H), 4.95 (t, J=7.0 Hz, 2H), 4.71 (dd, J=7.6, 5.6 Hz, 2H), 4.15 (s, 2H), 3.86 (d, J=1.4 Hz, 6H), 2.28 (s, 3H). ¹³C {¹H}NMR (CDCl₃, 126 MHz): δ 160.9, 150.6, 150.5, 146.1, 139.2, 129.3, 128.4, 128.2, 127.3, 126.6, 126.0, 125.7, 122.5, 122.4, 110.7, 78.4 (2C), 69.0, 62.4, 61.5, 29.4, 12.7. HRMS (ESI+) calcd. for C₂₂H₂₄NO₄: 366.169985. Found: 366.169403 (MH⁺).

### 5-((1,4-dimethoxy-3-methylnaphthalen-2-yl)methyl)-2-(oxetan-3-yloxy)pyrimidine (65)

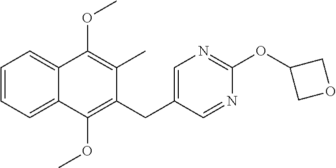 [SMILES: COc1c(C)c(Cc2cnc(OC3COC3)nc2)c(OC)c2ccccc12]

In a flame-dried tube was added 2-chloro-5-((1,4-dimethoxy-3-methylnaphthalen-2-yl)methyl)pyrimidine (100 mg, 0.304 mmol, 1 equiv.), oxetan-3-ol (39 μL, 0.608 mmol, 2 equiv.), anhydrous THF (1.5 mL) and potassium tert-butoxide (68.3 mg, 0.608 mmol, 2 equiv.). The tube was sealed and the mixture stirred at room temperature during 3 hours. The mixture was diluted with water addition and extracted three times with ethyl acetate. The crude residue was purified by flash chromatography on silica gel (cyclohexane/ethyl acetate, 1/1, v/v, UV) to afford 65 (82 mg, 74%) as a colorless oil. ¹H NMR (CDCl₃, 400 MHz): δ 8.29 (s, 2H), 8.11-8.03 (m, 2H), 7.55-7.47 (m, 2H), 5.53 (q, J=6.2 Hz, 1H), 4.96-4.89 (m, 2H), 4.76 (dd, J=8.0, 5.5 Hz, 2H), 4.12 (s, 2H), 3.89 (s, 3H), 3.86 (s, 3H), 2.30 (s, 3H). ¹³C {¹H} NMR (CDCl₃, 101 MHz): δ 162.9, 159.2 (2C), 150.9, 150.6, 128.4, 127.7, 127.3, 127.2, 126.3, 125.98, 125.95, 122.6, 122.5, 78.0 (2C), 70.1, 62.4, 61.6, 27.1, 12.9. HRMS (ESI+) calcd. for C₂₁H₂₃N₂O₄: 367.165324. Found: 367.165015 (MH⁺).

### Preparation of 5-((7-fluoro-1,4-dimethoxy-3-methylnaphthalen-2-yl)methyl)-2-(oxetan-3-yloxy)pyrimidine (66)

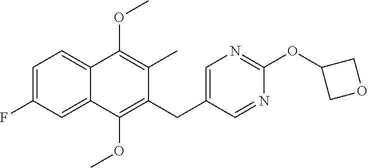 [SMILES: COc1c(C)c(Cc2cnc(OC3COC3)nc2)c(OC)c2cc(F)ccc12]

In a flame-dried tube was added 36 (92 mg, 0.27 mmol, 1 equiv.), oxetan-3-ol (34 μL, 0.53 mmol, 2 equiv.), anhydrous THF (1.3 mL) and potassium tert-butoxide (60 mg, 0.53 mmol, 2 equiv.). The tube was sealed and the mixture stirred at room temperature overnight. The mixture was diluted with water addition and extracted three times with ethyl acetate. The crude residue was purified by flash chromatography on silica gel (cyclohexane/ethyl acetate, 1/1, v/v, UV) to afford MR00331 (85 mg, 83%) as a colorless oil. ¹H NMR (CDCl₃, 400 MHz): δ 8.28 (s, 2H), 8.07 (dd, J=9.2, 5.5 Hz, 1H), 7.64 (dd, J=10.3, 2.5 Hz, 1H), 7.27 (ddd, J=9.2, 8.3, 2.6 Hz, 1H), 5.53 (p, J=6.0 Hz, 1H), 4.92 (t, J=7.1 Hz, 2H), 4.76 (dd, J=7.8, 5.6 Hz, 2H), 4.10 (s, 2H), 3.86 (s, 3H), 3.84 (s, 3H), 2.27 (s, 3H). ¹³C {¹H} NMR (CDCl₃, 101 MHz): δ 162.9, 161.1 (d, ¹J_{C-F}=245.9 Hz), 159.1 (2C), 151.0 (d, ⁵J_{C-F}=1.3 Hz), 150.1 (d, ⁴J_{C-F}=5.4 Hz), 128.7, 128.3 (d, ³J_{C-F}=8.7 Hz), 127.4, 125.44 (d, ⁵J_{C-F}=1.1 Hz), 125.36 (d, ³J_{C-F}=8.9 Hz), 125.2 (d, ⁴J_{C-F}=2.5 Hz), 116.6 (d, ²J_{C-F}=25.4 Hz), 106.4 (d, ²J_{C-F}=22.4 Hz), 77.9 (2C), 70.1, 62.3, 61.8, 27.2, 12.7. ¹⁹F NMR (CDCl₃, 377 MHz): δ-114.23 (ddd, J=10.1, 8.4, 5.6 Hz). HRMS (ESI+) calcd. for C₂₁H₂₂FN₂O₄: 385.155812. Found: 385.156295 (MH⁺).

### Preparation of 5-((7-fluoro-1,4-dimethoxy-3-methylnaphthalen-2-yl)methyl)-2-(oxetan-3-yloxy)pyridine (67)

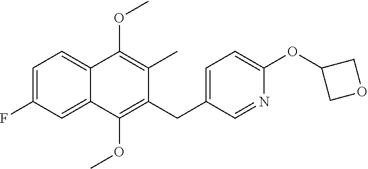 [SMILES: COc1c(C)c(Cc2ccc(OC3COC3)nc2)c(OC)c2cc(F)ccc12]

In a flame-dried tube was added 34 (83 mg, 0.25 mmol, 1 equiv.), oxetan-3-ol (32 μL, 0.50 mmol, 2 equiv.), anhydrous THF (1.3 mL) and potassium tert-butoxide (56 mg, 0.50 mmol, 2 equiv.). The tube was sealed and the mixture stirred at 55° C. overnight. The mixture was diluted with water addition and extracted three times with ethyl acetate. The crude residue was purified by flash chromatography on silica gel (cyclohexane/ethyl acetate, 8/2, v/v, UV) to afford MR00340 (74 mg, 77%) as a colorless oil. ¹H NMR (CDCl₃, 400 MHz): δ 8.07 (dd, J=9.2, 5.6 Hz, 1H), 7.86 (d, J=1.9 Hz, 1H), 7.66 (dd, J=10.4, 2.5 Hz, 1H), 7.36 (dd, J=8.5, 2.4 Hz, 1H), 7.29-7.22 (m, 1H), 6.67 (d, J=8.5 Hz, 1H), 5.55 (p, J=5.8 Hz, 1H), 4.95 (t, J=7.0 Hz, 2H), 4.74-4.67 (m, 2H), 4.13 (s, 2H), 3.84 (s, 3H), 3.83 (s, 3H), 2.26 (s, 3H). ¹³C {¹H} NMR (CDCl₃, 101 MHz): δ 161.0 (d, ¹J_{C-F}=245.4 Hz), 160.9, 150.8 (d, ⁵J_{C-F}=1.2 Hz), 150.0 (d, ⁴J_{C-F}=5.4 Hz), 146.0, 139.2, 130.0, 129.0, 128.30, 128.27 (d, ³J_{C-F}=8.6 Hz), 125.8 (d, ⁴J_{C-F}=2.5 Hz), 125.21 (d, ³J_{C-F}=8.8 Hz), 125.20, 116.2 (d, ²J_{C-F}=25.4 Hz), 110.8, 106.3 (d, ²J_{C-F}=22.3 Hz), 78.4 (2C), 69.0, 62.2, 61.6, 29.4, 12.6. ¹⁹F NMR (CDCl₃, 377 MHz): δ-114.64 (ddd, J=10.2, 8.4, 5.7 Hz). HRMS (ESI+) calcd. for C₂₂H₂₃FNO₄: 384.160563. Found: 384.161191 (MH⁺).

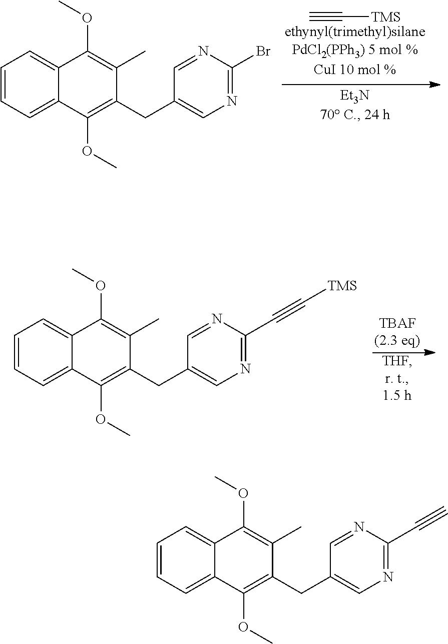

### Preparation of 5-[(1,4-dimethoxy-3-methylnaphthalen-2-yl)methyl]-2-[2-(trimethylsilyl)ethynyl]pyrimidine (68′)

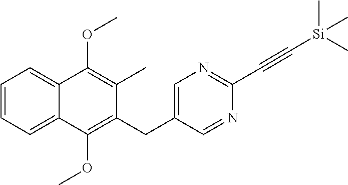 [SMILES: COc1c(C)c(Cc2cnc(C#C[Si](C)(C)C)nc2)c(OC)c2ccccc12]

In a flame dried sealable tube, under argon, to a solution of 33 (100 mg, 0.27 mmol, 1 equiv.) in triethylamine (8.5 mL) was added dichlorobis(triphenylphosphine)palladium(II) (11.9 mg, 0.017 mmol, 0.05 equiv.) followed by iodocopper (6.5 mg, 0.034 mmol, 0.1 equiv.). The mixture reaction was degassed with argon before the addition of ethynyl(trimethyl)silane (0.14 mL, 1.018 mmol, 3 equiv.). The tube was sealed and stirred at 70° C. for 24 hours. At room temperature, the mixture reaction was quenched with a solution of brine and water (1/1, v/v). The resulting aqueous layer was extracted three times with ethyl acetate. The combined organic layers were dried over MgSO₄ and the solvent was removed under reduced pressure. The crude residue was purified by flash chromatography on silica gel (cyclohexane/toluene, 8/2, v/v, UV) to afford 68′ (104 mg, 99% yield) as a brown oil. ¹H NMR (CDCl₃, 400 MHz): δ 8.49 (s, 2H), 8.13-8.03 (m, 2H), 7.58-7.47 (m, 2H), 4.21 (s, 2H), 3.85 (s, 3H), 3.85 (s, 3H), 2.26 (s, 3H), 0.27 (s, 9H). ¹³C {¹H} NMR (CDCl₃, 101 MHz): δ 157.0 (2C), 150.9, 150.8, 150.4, 132.8, 128.5, 127.2, 126.4, 126.3, 125.9, 125.8, 122.6, 122.5, 102.4, 93.8, 62.4, 61.6, 28.0, 12.9, 1.1 (3C). HRMS (ESI+) calcd. for C₂₃H₂₇SiN₂O₂: 391.183631. Found: 391.181585 (MH⁺).

### Preparation of 5-[(1,4-dimethoxy-3-methylnaphthalen-2-yl)methyl]-2-ethynylpyrimidine (68)

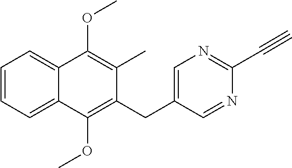 [SMILES: C#Cc1ncc(Cc2c(C)c(OC)c3ccccc3c2OC)cn1]

To a solution of 68′ (324 mg, 0.83 mmol, 1 equiv.) in THF (4.2 mL) was added dropwise a solution of tetra-n-butylammonium fluoride (602 mg, 1.91 mmol, 2.3 equiv.) in THF (4.2 mL). The reaction mixture was stirred at room temperature for 1.5 h. The mixture was quenched with a saturated aqueous solution of NH₄Cl. The aqueous layers were extracted three times with diethyl ether. The organic extracts were dried over MgSO₄ and concentrated under reduced pressure. The crude residue was purified by flash chromatography on silica gel (cyclohexane/ethyl acetate, 95/5, v/v, UV) to afford BDU0090 (163 mg, 62% yield) as a brown oil. ¹H NMR (CDCl₃, 400 MHz): δ 8.50 (s, 2H), 8.18-7.96 (m, 2H), 7.61-7.44 (m, 2H), 4.21 (s, 2H), 3.87 (s, 3H), 3.85 (s, 3H), 3.07 (s, 1H), 2.27 (s, 3H). ¹³C {¹H} NMR (CDCl₃, 101 MHz): δ 157.1, 150.9, 150.8, 150.0, 133.3, 128.5, 127.2, 126.4, 126.2, 126.0, 125.8, 122.6, 122.5, 81.9, 75.4, 62.4, 61.6, 28.1, 12.9. HRMS (ESI+) calcd. for C₂₀H₁₉N₂O₂: 319.144104. Found: 319.143171 (MH⁺).

### Preparation of Compounds of Formula (1) (by Oxidative Deprotection from the Compounds of Formula (11))

The compounds of formula (1) wherein R¹═H or F are prepared according to the following reaction scheme:

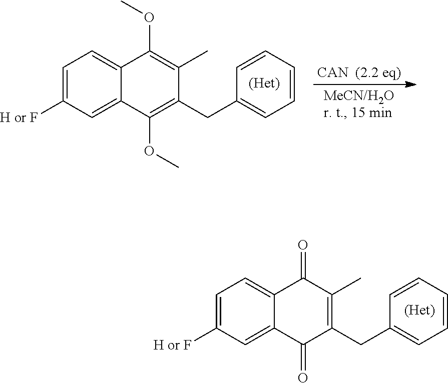

### General Procedure for the Oxidative Deprotection:

Suzuki coupling derivative (1 equiv) was dissolved in stirring acetonitrile. Then, at room temperature, CAN (2.1 equiv) dissolved in water was added drop by drop (ratio ACN/H₂O 3:1, 0.05M). The mixture was stirred at room temperature during 1 h. Then after TLC analysis showed complete conversion, the aqueous layer was extracted three times with dichloromethane. Combined organic layers were dried over MgSO4 and the solvent was removed under reduced pressure. Purification by silica gel chromatography was performed using the adequate eluent.

| Compound of formula (I) | Yield |
|---|---|
| 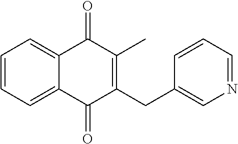 [SMILES: CC1=C(Cc2cccnc2)C(=O)c2ccccc2C1=O] | 72% |
| 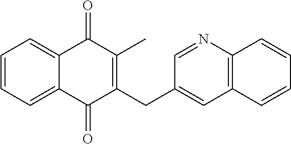 [SMILES: CC1=C(Cc2cnc3ccccc3c2)C(=O)c2ccccc2C1=O] | 86% |
| 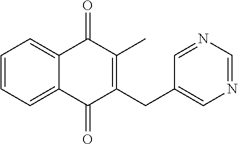 [SMILES: CC1=C(Cc2cncnc2)C(=O)c2ccccc2C1=O] | 88% |
| 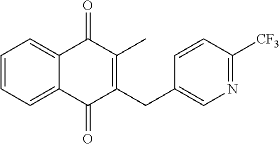 [SMILES: CC1=C(Cc2ccc(C(F)(F)F)nc2)C(=O)c2ccccc2C1=O] | 87% |
| 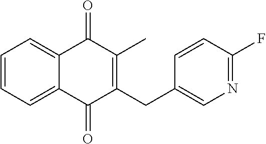 [SMILES: CC1=C(Cc2ccc(F)nc2)C(=O)c2ccccc2C1=O] | 97% |
| 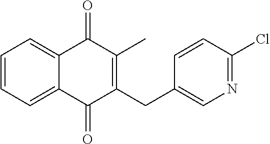 [SMILES: CC1=C(Cc2ccc(Cl)nc2)C(=O)c2ccccc2C1=O] | 100% |
| 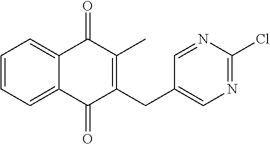 [SMILES: CC1=C(Cc2cnc(Cl)nc2)C(=O)c2ccccc2C1=O] | 93% |
| 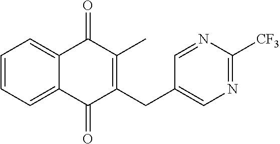 [SMILES: CC1=C(Cc2cnc(C(F)(F)F)nc2)C(=O)c2ccccc2C1=O] | 95% |
| 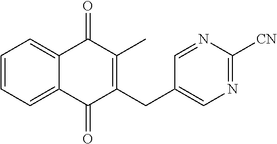 [SMILES: CC1=C(Cc2cnc(C#N)nc2)C(=O)c2ccccc2C1=O] | 87% |
| 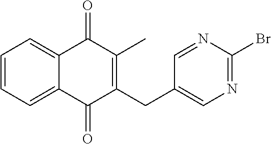 [SMILES: CC1=C(Cc2cnc(Br)nc2)C(=O)c2ccccc2C1=O] | 96% |
| 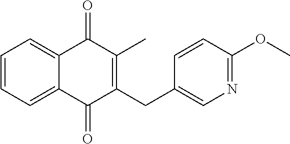 [SMILES: COc1ccc(CC2=C(C)C(=O)c3ccccc3C2=O)cn1] | 87% |
| 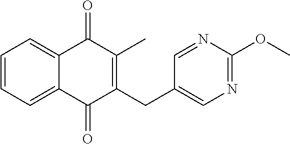 [SMILES: COc1ncc(CC2=C(C)C(=O)c3ccccc3C2=O)cn1] | 74% |
| 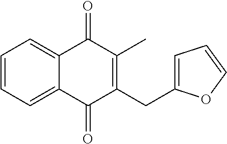 [SMILES: CC1=C(Cc2ccco2)C(=O)c2ccccc2C1=O] | 79% |

### Preparation of 2-methyl-3-(pyridin-3-ylmethyl)naphthalene-1,4-dione (1″)

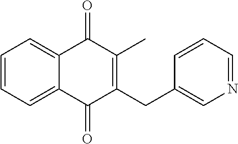 [SMILES: CC1=C(Cc2cccnc2)C(=O)c2ccccc2C1=O]

Eluant Cyclohexane:Ethyl acetate (1:1), yellow solid, 74% yield. ¹H NMR (400 MHz, CDCl₃) δ 8.54-8.51 (m, 1H), 8.43 (dd, J=4.8, 1.6 Hz, 1H), 8.16-7.97 (m, 2H), 7.70 (dd, J=5.8, 3.3 Hz, 2H), 7.54 (ddd, J=7.9, 2.4, 1.6 Hz, 1H), 7.18 (ddd, J=7.8, 4.8, 0.9 Hz, 1H), 4.01 (s, 2H), 2.25 (s, 3H). ¹³C NMR (101 MHz, CDCl₃) δ 185.16, 184.56, 150.05, 148.02, 144.83, 144.37, 136.22, 133.93, 133.78, 133.76, 132.16, 131.98, 126.61, 126.51, 123.69, 30.00, 13.44. HRMS (ESI) calcd. for C₁₇H₁₄NO₂: 264.1019. Found: 264.1018 (MH⁺).

### Preparation of 2-methyl-3-(quinolin-3-ylmethyl)naphthalene-1,4-dione (1′)

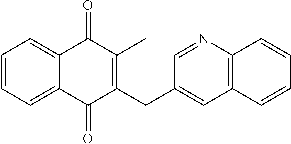 [SMILES: CC1=C(Cc2cnc3ccccc3c2)C(=O)c2ccccc2C1=O]

Eluant Cyclohexane:Ethyl acetate (1:1), yellow solid, 86% yield. ¹H NMR (400 MHz, CDCl₃) δ 8.87 (d, J=2.2 Hz, 1H), 8.14-8.06 (m, 2H), 8.05 (dd, J=8.5, 1.0 Hz, 1 H), 7.92 (dd, J=2.3, 1.0 Hz, 1H), 7.75-7.68 (m, 3H), 7.64 (ddd, J=8.4, 6.9, 1.5 Hz, 1H), 7.49 (ddd, J=8.1, 6.8, 1.2 Hz, 1H), 4.20 (s, 2H), 2.31 (s, 3H). ¹³C NMR (101 MHz, CDCl₃) δ 185.20, 184.58, 151.69, 147.08, 144.91, 144.38, 134.76, 133.80, 133.78, 132.19, 132.02, 131.12, 129.29, 129.20, 128.14, 127.57, 126.94, 126.65, 126.54, 30.19, 13.58. HRMS (ESI) calcd. for C₂₁H₁₆NO₂: 314.1176. Found: 314.1177 (MH⁺).

### Preparation of 2-methyl-3-(pyrimidin-5-ylmethyl)naphthalene-1,4-dione (2′)

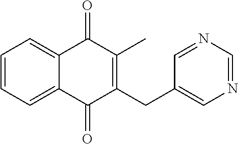 [SMILES: CC1=C(Cc2cncnc2)C(=O)c2ccccc2C1=O]

Eluant Cyclohexane:Ethyl acetate (1:1), yellow solid, 88% yield. ¹H NMR (500 MHz, CDCl₃) δ 9.04 (s, 1H), 8.64 (s, 2H), 8.10-7.98 (m, 2H), 7.76-7.61 (m, 2H), 3.98 (s, 2H), 2.27 (s, 3H). ¹³C NMR (126 MHz, CDCl₃) δ 184.80, 184.28, 157.22, 157.01, 145.07, 143.18, 133.96, 133.89, 132.05, 132.01, 131.79, 126.62, 27.82, 13.53. HRMS (ESI) calcd. for C₁₆H₁₃N₂O₂: 265.0972. Found: 265.0982 (MH⁺).

### Preparation of 2-methyl-3-((6-(trifluoromethyl)pyridin-3-yl)methyl) naphthalene-1,4-dione 3′)

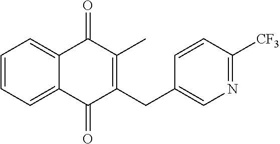 [SMILES: CC1=C(Cc2ccc(C(F)(F)F)nc2)C(=O)c2ccccc2C1=O]

Eluant Cyclohexane:Ethyl acetate (8:2), yellow solid, 87% yield. ¹H NMR (500 MHz, CDCl₃) δ 8.66 (d, J=2.2 Hz, 1H), 8.20-7.97 (m, 2H), 7.89-7.68 (m, 3H), 7.58 (dd, J=8.1, 0.8 Hz, 1H), 4.09 (s, 2H), 2.28 (s, 3H). ¹³C NMR (126 MHz, CDCl₃) δ 184.92, 184.43, 150.31, 146.62 (q, J=34.8 Hz), 145.23, 143.50, 137.47, 137.40, 133.99, 133.92, 132.11, 131.86, 126.66, 126.64, 121.62 (q, J=279.6 Hz), 120.54 (q, J=2.7 Hz), 30.00, 13.56. ¹⁹F NMR (471 MHz, CDCl₃) δ−67.81. HRMS (ESI) calcd. for C₁₈H₁₃F₃NO₂: 332.0893. Found: 332.0915 (MH⁺).

### Preparation of 2-((6-fluoropyridin-3-yl)methyl)-3-methylnaphthalene-1,4-dione (4′)

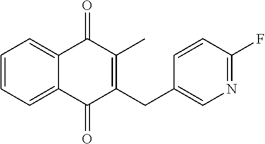 [SMILES: CC1=C(Cc2ccc(F)nc2)C(=O)c2ccccc2C1=O]

Eluant Cyclohexane:Ethyl acetate (8:2), yellow solid, 97% yield. ¹H NMR (500 MHz, CDCl₃) δ 8.13 (d, J=2.6 Hz, 1H), 8.08 (ddd, J=7.4, 5.8, 3.3 Hz, 2H), 7.72 (dd, J=5.8, 3.3 Hz, 2H), 7.67 (td, J=8.1, 2.6 Hz, 1H), 6.84 (dd, J=8.4, 3.0 Hz, 1H), 4.00 (s, 2H), 2.27 (s, 3H). ¹³C NMR (126 MHz, CDCl₃) δ 185.12, 184.59, 162.61 (d, J=238.4 Hz), 147.46 (d, J=14.7 Hz), 144.83, 144.17, 141.52 (d, J=7.8 Hz), 133.91, 133.86, 132.15, 131.95, 131.52 (d, J=4.7 Hz), 126.64, 126.60, 109.63 (d, J=37.5 Hz), 29.15, 13.45. ¹⁹F NMR (377 MHz, CDCl₃) δ−70.88 (d, J=7.7 Hz). HRMS (ESI) calcd. for C₁₇H₁₃FNO₂: 282.0925. Found: 282.0920 (MH⁺).

### Preparation of 2-((6-chloropyridin-3-yl)methyl)-3-methylnaphthalene-1,4-dione (5′)

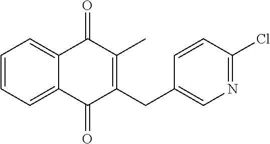 [SMILES: CC1=C(Cc2ccc(Cl)nc2)C(=O)c2ccccc2C1=O]

Eluant Cyclohexane:Ethyl acetate (8:2), yellow solid, 99% yield. ¹H NMR (500 MHz, CDCl₃) δ 8.30 (d, J=2.5 Hz, 1H), 8.11-7.96 (m, 2H), 7.75-7.63 (m, 2H), 7.51 (dd, J=8.2, 2.6 Hz, 1H), 7.20 (d, J=8.2 Hz, 1H), 3.97 (s, 2H), 2.25 (s, 3H). ¹³C NMR (126 MHz, CDCl₃) δ 184.98, 184.45, 149.79, 149.75, 144.92, 143.86, 139.11, 133.88, 133.82, 132.87, 132.08, 131.86, 126.59, 126.55, 124.33, 29.30, 13.45. HRMS (ESI) calcd. for C₁₇H₁₃ClNO₂: 298.0629. Found: 298.0648 (MH⁺).

### Preparation of 2-((2-chloropyrimidin-5-yl)methyl)-3-methylnaphthalene-1,4-dione (6)

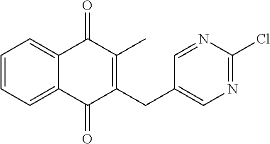 [SMILES: CC1=C(Cc2cnc(Cl)nc2)C(=O)c2ccccc2C1=O]

Eluant Cyclohexane:Ethyl acetate (7:3), yellow solid, 93% yield. ¹H NMR (400 MHz, CDCl₃) δ 8.54 (s, 2H), 8.20-7.92 (m, 2H), 7.85-7.57 (m, 2H), 3.96 (s, 2H), 2.29 (s, 3H). ¹³C NMR (101 MHz, CDCl₃) δ 184.58, 184.14, 159.74, 159.58, 145.09, 142.64, 133.97, 133.89, 131.93, 131.63, 130.56, 126.58, 126.55, 27.06, 13.49. HRMS (ESI) calcd. for C₁₆H₁₂ClN₂O₂: 299.0582. Found: 299.0596 (MH⁺).

### Preparation of 2-methyl-3-((2-(trifluoromethyl)pyrimidin-5-yl)methyl) naphthalene-1,4-dione (7)

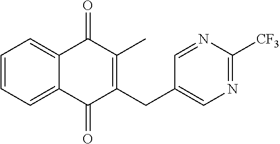 [SMILES: CC1=C(Cc2cnc(C(F)(F)F)nc2)C(=O)c2ccccc2C1=O]

Eluant Cyclohexane:Ethyl acetate (8:2), yellow solid, 3 95% yield. ¹H NMR (400 MHz, CDCl₃) δ 8.82 (s, 2H), 8.25-8.01 (m, 2H), 7.84-7.65 (m, 2H), 4.08 (s, 2H), 2.32 (s, 3H). ¹³C NMR (101 MHz, CDCl₃) δ 184.63, 184.23, 158.01, 155.31 (q, J=37.1 Hz), 145.49, 142.43, 134.44, 134.19, 134.08, 132.06, 131.72, 126.77, 126.73, 119.66 (q, J=275.1 Hz), 27.91, 13.68. ¹⁹F NMR (377 MHz, CDCl₃) δ−70.23. HRMS (ESI) calcd. for C₁₇H₁₂F₃N₂O₂: 333.0845. Found: 333.0868 (MH⁺).

### Preparation of 2-((6-methoxypyridin-3-yl)methyl)-3-methylnaphthalene-1,4-dione (8)

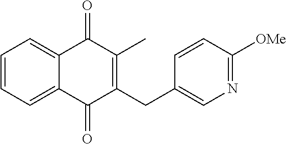 [SMILES: COc1ccc(CC2=C(C)C(=O)c3ccccc3C2=O)cn1]

Eluant Cyclohexane:Ethyl acetate (90:10), yellow solid, 87% yield. m.p: 120-121° C. ¹H NMR (400 MHz, CDCl₃) δ 8.11-7.95 (m, 3H), 7.67 (dd, J=5.8, 3.3 Hz, 2H), 7.50 (dd, J=8.6, 2.5 Hz, 21), 6.67 (d, J=8.6 Hz, 1H), 3.91 (s, 2H), 3.88 (s, 3H), 2.24 (s, 3H). ¹³C NMR (101 MHz, CDCl₃) δ 185.19, 184.61, 162.78, 145.90, 144.66, 144.43, 139.91, 133.70, 133.67, 132.11, 131.97, 126.66, 126.52, 126.43, 111.02, 53.79, 29.03, 13.31. HRMS (ESI) calcd. for C₁₈H₁₅NO₃: 294.112470. Found: 294.112517 (MH⁺).

### Preparation of 2-((2-methoxypyrimidin-5-yl)methyl)-3-methylnaphthalene-1,4-dione (9)

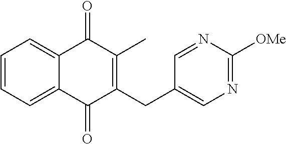 [SMILES: COc1ncc(CC2=C(C)C(=O)c3ccccc3C2=O)cn1]

Eluant Cyclohexane:Ethyl acetate (70:30), yellow solid, 74% yield. m.p: 161-162° C. ¹H NMR (400 MHz, CDCl₃) δ 8.40 (s, 2H), 8.10-7.96 (m, 2H), 7.67 (dd, J=5.8, 3.3 Hz, 2H), 3.92 (s, 3H), 3.87 (s, 2H), 2.25 (s, 3H). ¹³C NMR (101 MHz, CDCl₃) δ 184.91, 184.33, 164.61, 159.22, 144.51, 143.77, 133.81, 133.76, 132.01, 131.82, 126.51, 126.48, 124.86, 54.91, 26.75, 13.37. HRMS (ESI) calcd. for C₁₇H₁₅N₂O₃: 295.107719. Found: 295.108670 (MH⁺).

### Preparation of 2-(furan-2-ylmethyl)-3-methylnaphthalene-1,4-dione (10)

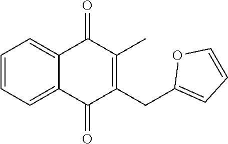 [SMILES: CC1=C(Cc2ccco2)C(=O)c2ccccc2C1=O]

Eluant Cyclohexane:Ethyl acetate (98:2), orange solid, 74% yield. m.p: 83-84° C. ¹H NMR (400 MHz, CDCl₃) δ 8.21-7.95 (m, 1H), 7.86-7.58 (m, 1H), 7.27 (dd, J=1.9, 0.9 Hz, OH), 6.26 (dd, J=3.2, 1.9 Hz, OH), 6.07 (dd, J=3.2, 0.9 Hz, OH), 4.03 (s, 1H), 2.28 (s, 2H). ¹³C NMR (101 MHz, CDCl₃) δ 185.34, 184.06, 151.27, 145.02, 142.41, 141.62, 133.62, 133.58, 132.21, 132.05, 126.56, 126.41, 110.55, 106.79, 25.57, 13.11.

### Preparation of 2-methyl-3-(thiophen-2-ylmethyl)naphthalene-1,4-dione (11)

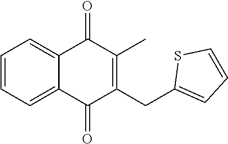 [SMILES: CC1=C(Cc2cccs2)C(=O)c2ccccc2C1=O]

m.p.=96-97° C. ¹H NMR (CDCl₃, 400 MHz): δ 8.11-8.03 (m, 2H), 7.71-7.65 (m, 2H), 7.11 (dd, J=4.5, 1.8 Hz, 1H), 6.89 (d, J=4.5 Hz, 2H), 4.18 (s, 2H), 2.29 (s, 3H). ¹³C NMR (CDCl₃, 101 MHz): 5 185.3, 184.2, 144.4, 144.1, 139.9, 133.61, 133.59,132.1,132.0, 126.9, 126.5, 126.4, 125.8, 124.2, 27.0, 13.1. HRMS calculated for C₁₆H₁₂NaO₂S [M+Na]⁺: 291.045021. Found 291.045521.

### Preparation of 5-((3-methyl-1,4-dioxo-1,4-dihydronaphthalen-2-yl)methyl)pyrimidine-2-carbonitrile (12)

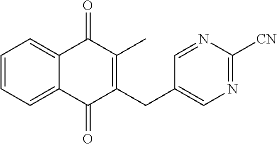 [SMILES: CC1=C(Cc2cnc(C#N)nc2)C(=O)c2ccccc2C1=O]

Eluant Cyclohexane:Ethyl acetate (7:3), yellow solid, 87% yield. ¹H NMR (400 MHz, CDCl₃) δ 8.76 (s, 2H), 8.10-7.99 (m, 2H), 7.81-7.61 (m, 2H), 4.06 (s, 2H), 2.31 (s, 3H). ¹³C NMR (101 MHz, CDCl₃) δ 184.50, 184.17, 158.11, 145.67, 143.40, 142.05, 135.19, 134.23, 134.09, 132.00, 131.62, 126.77, 126.69, 115.68, 28.20, 13.72. HRMS (ESI) calcd. for C₁₇H₁₂N₃O₂: 290.0924. Found: 290.0918 (MH⁺).

### Preparation of 2-((2-bromopyrimidin-5-yl)methyl)-3-methylnaphthalene-1,4-dione (13)

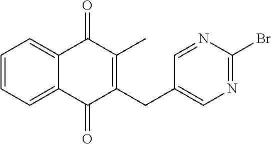 [SMILES: CC1=C(Cc2cnc(Br)nc2)C(=O)c2ccccc2C1=O]

Eluant Cyclohexane:Ethyl acetate (75:25), yellow solid, 96% yield. m.p: 165-166° C. ¹H NMR (400 MHz, CDCl₃) 5 8.48 (s, 2H), 8.11-8.00 (m, 2H), 7.80-7.60 (m, 2H), 3.94 (s, 2H), 2.28 (s, 3H). ¹³C NMR (101 MHz, CDCl₃) δ 184.67, 184.23, 159.56, 151.26, 145.20, 142.67, 134.09, 134.00, 132.03, 131.73, 131.14, 126.69, 126.66, 27.24, 13.61. HRMS (ESI) calcd. for C₁₆H₁₂BrN₂O₂: 343.0077. Found: 343.0099 (MH⁺).

### Preparation of 2-((2-aminopyrimidin-5-yl)methyl)-3-methylnaphthalene-1,4-dione (46)

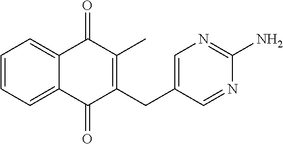 [SMILES: CC1=C(Cc2cnc(N)nc2)C(=O)c2ccccc2C1=O]

46 was isolated by purification by flash chromatography on silica gel (dichloromethane/methanol, 9/1, v/v, UV) with 60% yield as an orange solid. ¹H NMR (CDCl₃, 500 MHz): δ 8.12 (s, 2H), 8.01-7.96 (m, 2H), 7.84-7.80 (m, 2H), 6.46 (s, 2H), 3.73 (s, 2H), 2.18 (s, 3H). ¹³C {¹H} NMR (CDCl₃, 126 MHz): δ 184.6, 184.2, 162.4, 144.8, 143.8, 133.9, 133.8, 131.7, 131.5, 125.9, 125.8, 119.5, 26.1, 13.0. HRMS (ESI+) calcd. for C₁₆H₁₄N₃O₂: 290.1081. Found: 280.1081 (MH⁺). M.p.=degradation after 200° C.

### Preparation of 6-fluoro-2-methyl-3-((6-(trifluoromethyl)pyridin-3-yl)methyl)naphthalene-1,4-dione (47)

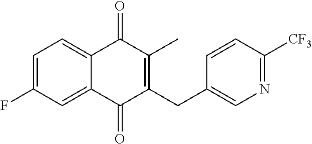 [SMILES: CC1=C(Cc2ccc(C(F)(F)F)nc2)C(=O)c2cc(F)ccc2C1=O]

47 was isolated by purification by flash chromatography on silica gel (cyclohexane/ethyl acetate, 7/3 then pentane/diethyl ether, 8/2, v/v, UV) with 61% yield as a yellow solid. ¹H NMR (CDCl₃, 400 MHz): δ 8.65 (s, 1H), 8.13 (dd, J=8.5, 5.2 Hz, 1H), 7.73 (d, J=8.2 Hz, 1H), 7.70 (dd, J=8.5, 2.5 Hz, 1H), 7.58 (d, J=7.8 Hz, 1H), 7.37 (td, J=8.3, 2.4 Hz, 1H), 4.09 (s, 2H), 2.29 (s, 3H). ¹³C {¹H} NMR (CDCl₃, 101 MHz): δ 183.6, 183.4, 166.2 (d, ¹J_{C-F}=257.5 Hz), 150.5, 146.7 (q, ²J_{C-F}=34.9 Hz), 145.5, 143.6 (d, ⁵J_{C-F}=1.6 Hz), 137.5, 137.2, 134.4 (d, ³J_{C-F}=7.9 Hz), 130.0 (d, ³J_{C-F}=8.9 Hz), 128.7 (d, ⁴J_{C-F}=3.2 Hz), 121.6 (q, ¹J_{C-F}=273.7 Hz), 121.2 (d, ²J_{C-F}=22.5 Hz), 120.6 (d, ⁴J_{C-F}=2.2 Hz), 113.4 (d, ²J_{C-F}=23.5 Hz), 30.2, 13.7. ¹⁹F NMR (CDCl₃, 377 MHz): δ-67.80, −101.84 (td, J=8.2, 5.3 Hz). HRMS (ESI+) calcd. for C₁₈H₁₂F₄NO₂: 350.079868. Found: 350.079641 (MH⁺). M.p.=122-124° C.

### Preparation of 3-(benzo[d]thiazol-5-ylmethyl)-6-fluoro-2-methyl-naphthalene-1,4-dione (48)

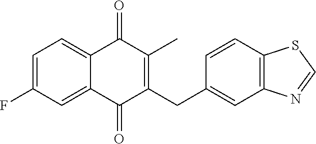 [SMILES: CC1=C(Cc2ccc3scnc3c2)C(=O)c2cc(F)ccc2C1=O]

48 was isolated by purification by flash chromatography on silica gel (cyclohexane/ethyl acetate, 7/3, v/v, UV) and by recrystallization in ethanol with 91% yield as a yellow solid. ¹H NMR (CDCl₃, 400 MHz): δ 9.04 (s, 1H), 8.13 (dd, J=8.5, 5.2 Hz, 1H), 7.95 (s, 1H), 7.89 (d, J=8.2 Hz, 1H), 7.72 (dd, J=8.5, 2.5 Hz, 1H), 7.46-7.30 (m, 2H), 4.20 (s, 2H), 2.29 (s, 3H). ¹³C {¹H} NMR (CDCl₃, 101 MHz): δ 184.0, 183.6, 166.1 (d, ¹J_{C-F}=256.9 Hz), 155.3, 154.2, 145.2 (d, ⁵J_{C-F}=1.8 Hz), 145.2, 136.5, 134.7 (d, ³J_{C-F}=7.9 Hz), 132.4, 129.8 (d, ³J_{C-F}=8.8 Hz), 128.8 (d, ⁴J_{C-F}=3.2 Hz), 126.8, 123.1, 122.2, 120.9 (d, ²J_{C-F}=22.6 Hz), 113.3 (d, ²J_{C-F}=23.5 Hz), 32.4, 13.6. ¹⁹F NMR (CDCl₃, 377 MHz): δ-102.4 (td, J=8.3, 5.3 Hz). HRMS (ESI+) calcd. for C₁₉H₁₃FNO₂S: 338.064554. Found: 338.063505 (MH⁺). M.p.=109-111° C.

### Preparation of 2-(benzo[d]thiazol-5-ylmethyl)-3-methylnaphthalene-1,4-dione (49)

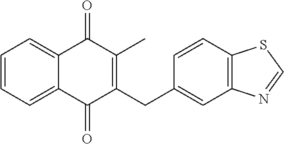 [SMILES: CC1=C(Cc2ccc3scnc3c2)C(=O)c2ccccc2C1=O]

49 was isolated by purification by flash chromatography on silica gel (cyclohexane/ethyl acetate, 8/2, v/v, UV). ¹H NMR (CDCl₃, 400 MHz): δ 8.97 (s, 1H), 8.06 (dt, J=5.7, 3.1 Hz, 2H), 7.95 (s, 1H), 7.85 (d, J=8.2 Hz, 1H), 7.83-7.57 (m, 1H), 7.36 (d, J=8.1 Hz, 2H), 4.18 (s, 2H), 2.27 (s, 3H). ¹³C {¹H} NMR (CDCl₃, 101 MHz): δ 185.3, 184.6, 154.8, 153.9, 145.0, 144.8, 136.6, 133.6 (2C), 132.1 (2C), 132.0, 126.7, 126.5, 126.4, 123.1, 122.0, 32.3, 13.4. HRMS (ESI+) calcd. for C₁₉H₁₃NNaO₂S: 342.055920. Found: 342.055527 (MNa⁺). M.p.=148-149° C.

### Preparation of 2-methyl-3-((6-(oxetan-3-yloxy)pyridin-3-yl)methyl) naphthalene-1,4-dione (50)

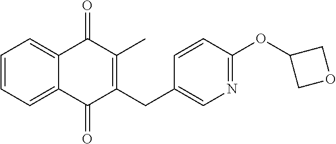 [SMILES: CC1=C(Cc2ccc(OC3COC3)nc2)C(=O)c2ccccc2C1=O]

50 was isolated by solubilisation in ethanol with 85% yield as a yellow solid. ¹H NMR (CD₃OD, 400 MHz): δ 8.47 (s, 1H), 8.36 (dd, J=9.1, 1.8 Hz, 1H), 8.12-8.04 (m, 2H), 7.82-7.75 (m, 2H), 7.42 (d, J=9.0 Hz, 1H), 5.48 (ddt, J=9.6, 6.9, 2.6 Hz, 1H), 4.97 (dd, J=12.3, 9.8 Hz, 1H), 4.82 (dd, d apparent because of HDO peak, J=7.0 Hz, 1H), 4.13 (s, 2H), 4.03 (dd, J=13.1, 2.4 Hz, 1H), 3.80 (dd, J=13.1, 2.9 Hz, 1H), 2.31 (s, 3H). ¹³C {¹H} NMR (CD₃OD, 101 MHz): δ 185.9, 185.6, 161.1, 150.6, 147.3, 143.4, 137.8, 135.0, 134.9, 133.4, 133.1, 132.4, 127.3, 127.2, 111.0, 86.2, 62.4, 53.2, 29.7, 13.4. H RMS (ESI+) calcd. for C₂₀H₁₈NO₄: 336.123034 Found: 336.123633 (MH⁺). M.p.=125-127° C.

### Preparation of 2-(benzo[d]thiazol-6-ylmethyl)-3-methylnaphthalene-1,4-dione (51)

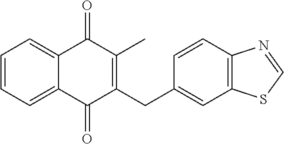 [SMILES: CC1=C(Cc2ccc3ncsc3c2)C(=O)c2ccccc2C1=O]

51 was isolated by purification by flash chromatography on silica gel (cyclohexane/ethyl acetate, 8/2, v/v, UV) with 78% yield as a yellow solid. ¹H NMR (CDCl₃, 400 MHz): δ 8.95 (s, 1H), 8.15-8.06 (m, 2H), 8.04 (d, J=8.3 Hz, 1H), 7.81 (s, 1H), 7.71 (dd, J=5.7, 3.3 Hz, 2H), 7.42 (d, J=7.8 Hz, 1H), 4.19 (s, 2H), 2.29 (s, 3H). ¹³C {¹H} NMR (CDCl₃, 101 MHz): δ 185.3, 184.7, 154.3, 152.4, 144.9, 144.7, 135.9, 134.8, 133.65, 133.64, 132.1, 132.0, 127.3, 126.6, 126.4, 123.6, 121.5, 32.4, 13.5. HRMS (ESI+) calcd. for C₁₉H₁₄NO₂S: 320.074032 Found: 320.074032 (MH⁺). M.p.=146-147° C.

### Preparation of 3-(benzo[d]thiazol-6-ylmethyl)-6-fluoro-2-methylnaphthalene-1,4-dione (52)

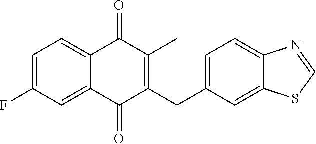 [SMILES: CC1=C(Cc2ccc3ncsc3c2)C(=O)c2cc(F)ccc2C1=O]

52 was isolated by purification by flash chromatography on silica gel (cyclohexane/ethyl acetate, 8/2, v/v, UV) as a yellow solid with 42% yield. ¹H NMR (CDCl₃, 500 MHz): δ 8.96 (s, 1H), 8.13 (dd, J=8.6, 5.2 Hz, 1H), 8.04 (d, J=7.5 Hz, 1H), 7.80 (s, 1H), 7.73 (dd, J=8.6, 2.6 Hz, 1H), 7.44-7.34 (m, 2H), 4.17 (s, 2H), 2.29 (s, 3H). ¹³C {¹H} NMR (CDCl₃, 126 MHz): δ 184.0, 183.7, 166.1 (d, ¹J_{C-F}=257.0 Hz), 154.4, 152.2, 145.18, 145.17, 145.1, 135.8, 134.6 (d, ³J_{C-F}=7.8 Hz), 129.8 (d, ³J_{C-F}=8.8 Hz), 128.8 (d, ⁴J_{C-F}=3.1 Hz), 127.3, 123.8, 121.6, 120.9 (d, ²J_{C-F}=22.5 Hz), 113.4 (d, ²J_{C-F}=23.5 Hz), 32.6, 13.6. ¹⁹F NMR (CDCl₃, 377 MHz): δ-102.28 (td, J=8.2, 5.3 Hz). HRMS (ESI+) calcd. for C₁₉H₁₃FNO₂S: 338.064554 Found: 338.064817 (MH⁺). M.p.=170-172° C.

### Preparation of 2-methyl-3-((2-(oxetan-3-yloxy)pyrimidin-5-yl)methyl)naphthalene-1,4-dione (53)

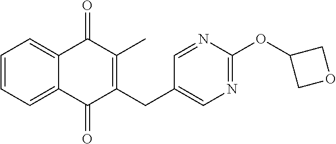 [SMILES: CC1=C(Cc2cnc(OC3COC3)nc2)C(=O)c2ccccc2C1=O]

53 was isolated after precipitation in dichloromethane with 39% yield as a yellow solid. ¹ H NMR (DMSO-d₆, 400 MHz): δ 9.24 (d, J=2.5 Hz, 1H), 9.04 (d, J=2.4 Hz, 1H), 8.07-7.95 (m, 2H), 7.90-7.82 (m, 2H), 5.45 (ddt, J=9.7, 6.9, 3.2 Hz, 1H), 4.88 (dd, J=12.7, 9.8 Hz, 1H), 4.65 (dd, J=12.7, 7.1 Hz, 1H), 4.09 (s, 2H), 3.88 (dd, J=13.0, 2.5 Hz, 1H), 3.74 (dd, J=13.0, 3.7 Hz, 1H), 2.21 (s, 3H). ¹³C {¹H} NMR (DMSO-d₆, 101 MHz): δ 184.4, 183.7, 169.3, 159.7, 148.0, 146.0, 141.1, 134.1, 134.0, 131.8, 131.5, 127.0, 126.0, 83.6, 60.7, 50.8, 25.9, 13.2. HRMS (ESI+) calcd. for C₁₉H₁₇N₂O₄: 337.118684 Found: 337.118684 (MH⁺). M.p.=degradation after 165° C.

### Preparation of 6-fluoro-2-methyl-3-((2-(oxetan-3-yloxy)pyrimidin-5-yl)methyl)naphthalene-1,4-dione (54)

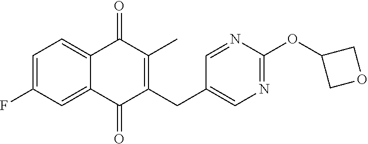 [SMILES: CC1=C(Cc2cnc(OC3COC3)nc2)C(=O)c2cc(F)ccc2C1=O]

54 was isolated after precipitation in dichloromethane with 98% yield as a yellow solid. ¹H NMR (DMSO-d₆, 400 MHz): δ9.23 (d, J=2.5 Hz, 1H), 9.02 (d, J=2.3 Hz, 1H), 8.14 (dd, J=9.3, 5.4 Hz, 1H), 7.72 (ddd, J=8.8, 6.2, 2.7 Hz, 2H), 5.45 (ddt, J=9.9, 6.8, 3.2 Hz, 1H), 4.86 (dd, J=12.6, 9.8 Hz, 1H), 4.64 (dd, J=12.7, 7.1 Hz, 1H), 4.09 (s, 2H), 3.88 (dd, J=13.0, 2.5 Hz, 1H), 3.73 (dd, J=13.0, 3.7 Hz, 1H), 2.21 (s, 3H). ¹³C {¹H}NMR (DMSO-d₆, 101 MHz): δ 183.2, 182.7 (d, ⁴J_{C-F}=1.2 Hz), 169.4, 165.3 (d, ¹J_{C-F}=254.0 Hz), 159.7, 148.0, 146.3, 141.3 (d, ⁵J_{C-F}=1.8 Hz), 134.3 (d, ³J_{C-F}=7.9 Hz), 129.7 (d, ³J_{C-F}=9.2 Hz), 128.8 (d, ⁴J_{C-F}=3.0 Hz), 126.8, 121.2 (d, ²J_{C-F}=22.6 Hz), 112.4 (d, ²J_{C-F}=23.4 Hz), 83.7, 60.7, 50.8, 26.0, 13.2. ¹⁹F NMR (CD₃OD, 377 MHz): δ-105.18 (td, J=8.6, 5.3 Hz). HRMS (ESI+) calcd. for C₁₉H₁₆FN₂O₄: 355.108862 Found: 355.107660 (MH⁺). M.p.=degradation after 165° C.

### Preparation of 2-[(6-aminopyridin-3-yl)methyl]-3-methyl-1,4-dihydronaphthalene-1,4-dione (55)

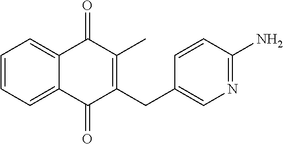 [SMILES: CC1=C(Cc2ccc(N)nc2)C(=O)c2ccccc2C1=O]

0 55 was isolated by purification by flash chromatography on silica gel (ethyl acetate) with 68% yield as an orange solid. ¹H NMR (400 MHz, CDCl₃) δ 8.08 (dt, J=5.9, 3.0 Hz, 2H), 7.95 (s, 1H), 7.76-7.60 (m, 2H), 7.35 (dd, J=8.5, 2.3 Hz, 1H), 6.44 (d, J=8.5 Hz, 1H), 4.59 (s, 2H), 3.86 (s, 2H), 2.26 (s, 3H). ¹³C {¹H} NMR (101 MHz, CDCl₃) δ 185.3, 184.7, 156.8, 146.6, 144.9, 144.2, 138.9, 133.6, 132.1, 131.9, 126.4, 126.3, 109.1, 28.9, 13.2.

### Preparation of 2-[(2-ethynylpyrimidin-5-yl)methyl]-3-methyl-1,4-dihydronaphthalene-1,4-dione (56)

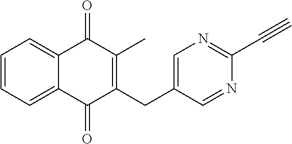 [SMILES: C#Cc1ncc(CC2=C(C)C(=O)c3ccccc3C2=O)cn1]

56 was isolated by purification by flash chromatography on silica gel (cyclohexane/ethyl acetate, 8/2, v/v, UV) as an orange solid with 70% yield. ¹H NMR (CDCl₃, 500 MHz): δ 8.63 (s, 2H), 8.13-7.99 (m, 2H), 7.81-7.61 (m, 2H), 3.99 (s, 2H), 3.09 (s, 1H), 2.28 (s, 3H). ¹³C {¹H} NMR (CDCl₃, 126 MHz): δ 184.8, 184.3, 157.4 (2C), 150.5, 145.3, 142.8, 134.1, 134.0, 132.1, 131.8, 131.4, 126.7 (2C), 81.7, 75.9, 27.9, 13.6. HRMS (ESI+) calcd. For C₁₈H₁₃N₂O₂: 289.097154 Found: 289.096682 (MH⁺). M.p.=160-162° C.

### Preparation of Compounds of Formula (II) (by Suzuki Reaction Between 6-Fluoro-3-Chloromethyl-1,4-Dimethoxynaphtalene and Boronic Acids of Formula (III))

The compounds of formula (II) wherein R¹═F are prepared according to the following reaction scheme:

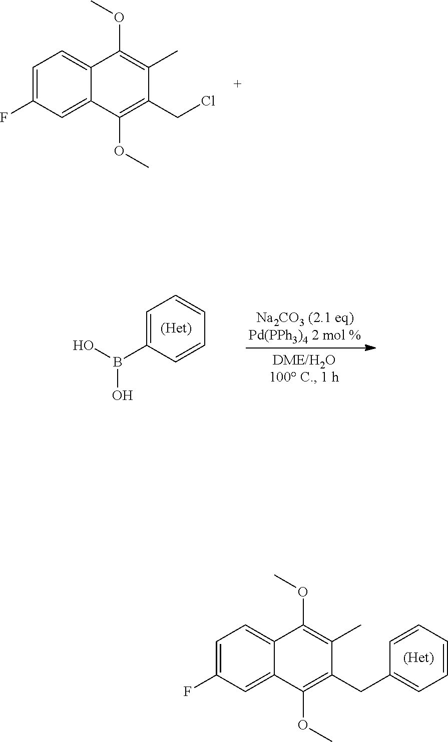

### Synthesis of 6-fluoro precursors

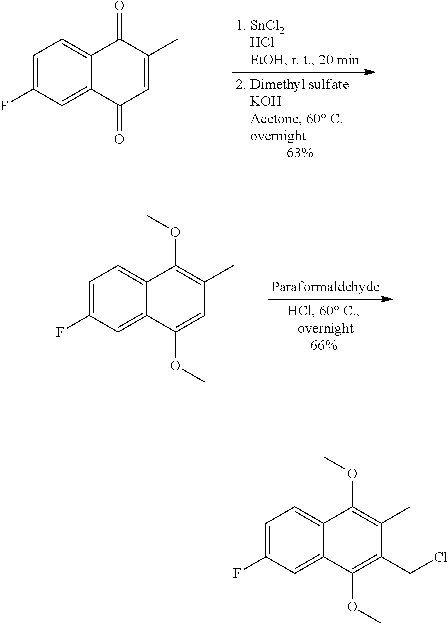 [SMILES: CC1=CC(=O)c2cc(F)ccc2C1=O ; COc1cc(C)c(OC)c2ccc(F)cc12 ; COc1c(C)c(CCl)c(OC)c2cc(F)ccc12]

### Preparation of 6-fluoro-1,4-dimethoxy-2-methylnaphthalene

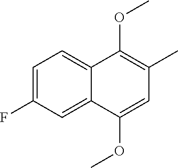 [SMILES: COc1cc(C)c(OC)c2ccc(F)cc12]

According to the previously published method (T. Mller, L. Johann, B. Jannack, M. Bruckner, D. A. Lanfranchi, H. Bauer, C. Sanchez, V. Yardleyll, C. Deregnaucourt, J. Schrevel, M. Lanzer, R. H. Schirmer, E. Davioud-Charvet, J. Am. Chem. Soc. 2011, 133, 30, 11557-11571), this compound was purified by flash chromatography on silica gel (Toluene, UV) with 63% yield (beige solid). m.p.=52-53° C. ¹H NMR (CDCl₃, 400 MHz): δ 8.07 (dd, J=9.2, 5.6 Hz, 1H), 7.88 (dd, J=10.6, 2.7 Hz, 1H), 7.32 (ddd, J=9.2, 8.3, 2.7 Hz, 1H), 6.64 (s, 1H), 3.95 (s, 3H), 3.89 (s, 3H), 2.48 (s, 3H). ¹³C NMR (CDCl₃, 101 MHz): δ 160.4 (d, ¹J_{C-F}=243.9 Hz), 150.9 (d, ⁴J_{C-F}=5.1 Hz), 147.1 (d, ⁵J_{C-F}=1.4 Hz), 126.1 (d, ³J_{C-F}=8.8 Hz), 125.8 (d, ⁵J_{C-F}=0.8 Hz), 124.8 (d, ⁴J_{C-F}=2.5 Hz), 124.2 (d, ³J_{C-F}=8.7 Hz), 116.3 (d, ²J_{C-F}=25.2 Hz), 108.0, 106.3 (d, ²J_{C-F}=22.5 Hz), 61.2, 55.5, 16.1. ¹⁹F NMR (CDCl₃, 377 MHz): δ-116.24 (ddd, J=10.3, 8.4, 5.5 Hz). HRMS calculated for C₁₃H₁₄FO₂ [M+H]⁺: 221.097234. Found 221.097121.

### Preparation of 3-(chloromethyl)-6-fluoro-1,4-dimethoxy-2-methylnaphthalene

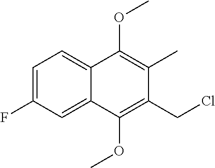 [SMILES: COc1c(C)c(CCl)c(OC)c2cc(F)ccc12]

A solution of 6-fluoro-1,4-dimethoxy-2-methylnaphthalene (250 mg, 1.14 mmol, 1 eq) and paraformaldehyde (538 mg, 17.03 mmol, 15 eq) in 37% aqueous hydrochloric acid (9 mL) was stirred at 60° C. overnight. The reaction mixture was cooled down, diluted with water, and extracted with ethyl acetate. The organic phase was dried over MgSO₄ and concentrated under reduced pressure. The crude residue was purified by flash chromatography on silica gel (Toluene/cyclohexane, 9/1, v/v, UV) to afford the expected product (200 mg, 66%, white solid). m.p.=100-102° C. ¹H NMR (CDCl₃, 400 MHz): δ 8.07 (dd, J=9.2, 5.5 Hz, 1H), 7.68 (dd, J=10.2, 2.5 Hz, 1H), 7.29 (ddd, J=9.2, 8.2, 2.6 Hz, 1H), 4.89 (s, 2H), 4.02 (s, 3H), 3.87 (s, 3H), 2.52 (s, 3H). ¹³C NMR (CDCl₃, 101 MHz): δ 161.1 (d, ¹J_{C-F}=246.0 Hz), 150.9 (d, ⁴J_{C-F}=5.4 Hz), 150.7 (d, ⁵J_{C-F}=1.3 Hz), 128.2 (d, ³J_{C-F}=8.7 Hz), 128.0, 126.3 (d, ⁵J_{C-F}=0.6 Hz), 125.6 (d, ⁴J_{C-F}=2.5 Hz), 125.3 (d, ³J_{C-F}=8.8 Hz), 117.1 (d, ²J_{C-F}=25.3 Hz), 106.7 (d, ²J_{C-F}=22.4 Hz), 63.1, 61.6, 38.9. ¹⁹F NMR (CDCl₃, 377 MHz): δ-114.27 (m). HRMS calculated for C₁₄H₁₅CIFO₂ [M+H]⁺: 269.073912. Found 269.073883.

### General procedure for the Suzuki coupling between 2-(chloromethyl)-6-fluoro-1,4-dimethoxy-3-methylnaphthalene and heteroarylboronic acid

In a flame dried sealable tube, under argon, 2-(chloromethyl)-6-R-1,4-dimethoxy-2-methylnaphthalene (1 eq), boronic acid (1.2 eq), sodium carbonate (2.1 eq) were introduced successively in a mixture of dimethoxyethane and water (ratio 2/1, v/v). The solvent was degassed and tetrakis(triphenylphosphine)palladium (0.02 eq) was added in the solution. The tube was sealed and stirred at 100° C. for 1 h. The reaction mixture was diluted with water and extracted 3 times with dichloromethane. The organic layer was washed with brine, dried over MgSO₄, and concentrated under reduced pressure. The crude residue was purified by flash chromatography on silica gel to afford the expected product.

| Boronic acid of formula (III) | Compound of formula (II) | Yield |
|---|---|---|
| 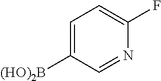 | 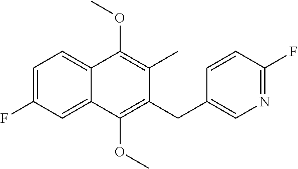 [SMILES: COc1c(C)c(Cc2ccc(F)nc2)c(OC)c2cc(F)ccc12] | 71% |
| 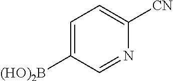 [SMILES: N#Cc1ccc(B(O)O)cn1] | 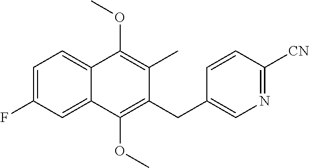 [SMILES: COc1c(C)c(Cc2ccc(C#N)nc2)c(OC)c2cc(F)ccc12] | 70% |
| 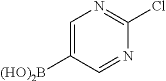 | 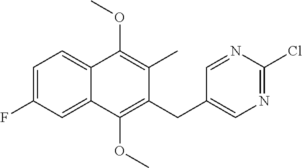 [SMILES: COc1c(C)c(Cc2cnc(Cl)nc2)c(OC)c2cc(F)ccc12] | 70% |
| 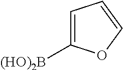 | 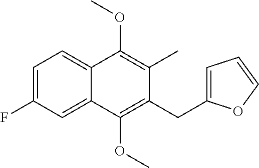 [SMILES: COc1c(C)c(Cc2ccco2)c(OC)c2cc(F)ccc12] | 83% |
| 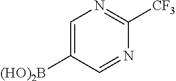 [SMILES: OB(O)c1cnc(C(F)(F)F)nc1] | 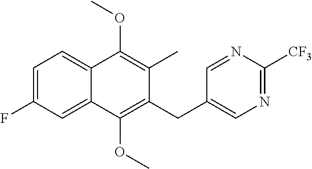 [SMILES: COc1c(C)c(Cc2cnc(C(F)(F)F)nc2)c(OC)c2cc(F)ccc12] | 73% |

### Preparation of 2-fluoro-5-((7-fluoro-1,4-dimethoxy-3-methylnaphthalen-2-yl)methyl)pyridine (34)

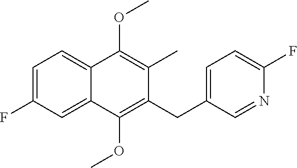 [SMILES: COc1c(C)c(Cc2ccc(F)nc2)c(OC)c2cc(F)ccc12]

This compound was isolated by purification by flash chromatography on silica gel (Cyclohexane/Ethyl acetate, 9/1, v/v, UV) with 71% yield (colourless oil). ¹H NMR (CDCl₃, 400 MHz): δ 8.10 (dd, J=9.3, 5.7 Hz, 1H), 8.07 (s, 1H), 7.69 (dd, J=10.3, 2.5 Hz, 1H), 7.52 (td, J=8.2, 2.3 Hz, 1H), 7.28 (td, J=9.2, 2.6 Hz, 1H), 6.80 (dd, J=8.4, 2.9 Hz, 1H), 4.22 (s, 2H), 3.86 (d, J=1.7 Hz, 6H), 2.27 (s, 3H). ¹³C NMR (CDCl₃, 101 MHz): δ 162.4 (d, ¹J_{C-F}=237.3 Hz), 161.0 (d, ¹J_{C-F}=246.4 Hz), 150.9 (d, ⁵J_{C-F}=2.0 Hz), 150.1 (d, ⁴J_{C-F}=5.4 Hz), 146.9 (d, ³J_{C-F}=14.5 Hz), 141.0 (d, ³J_{C-F}=7.7 Hz), 133.3 (d, ⁴J_{C-F}=4.6 Hz), 129.4, 128.3 (d, ³J_{C-F}=8.7 Hz), 125.6 (d, ⁴J_{C-F}=2.5 Hz), 125.3, 125.2 (d, ³J_{C-F}=9.1 Hz), 116.3 (d, ²J_{C-F}=25.4 Hz), 109.3 (d, ²J_{C-F}=37.5 Hz), 106.3 (d, ²J_{C-F}=22.4 Hz), 62.2, 61.6, 29.4, 12.6. ¹⁹F NMR (CDCl₃, 377 MHz): δ-114.42 (ddd, J=10.1, 8.4, 5.7 Hz). HRMS calculated for C₁H₁₈F₂NO₂ [M+H]⁺: 330.130012. Found 330.131085.

### Preparation of 5-((7-fluoro-1,4-dimethoxy-3-methylnaphthalen-2-yl)methyl)picolinonitrile (35)

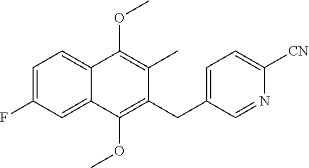 [SMILES: COc1c(C)c(Cc2ccc(C#N)nc2)c(OC)c2cc(F)ccc12]

This compound was isolated by purification by flash chromatography on silica gel (Cyclohexane/Ethyl acetate, 4/1, v/v, UV) with 70% yield (white solid). m.p.=132-134° C. ¹H NMR (CDCl₃, 400 MHz): δ68.60 (d, J=1.36 Hz, 1H), 8.08 (dd, J=9.2, 5.6 Hz, 1H), 7.65 (dd, J=10.3, 2.5 Hz, 1H), 7.59-7.45 (m, 2H), 7.32-7.23 (m, 1H), 4.29 (s, 2H), 3.91-3.79 (m, 6H), 2.23 (s, 3H). ¹³C NMR (CDCl₃, 101 MHz): δ 161.1 (d, ¹J_{C-F}=246.1 Hz), 151.3, 151.0 (d, ⁵J_{C-F}=1.3 Hz), 150.2 (d, ⁴J_{C-F}=5.4 Hz), 140.3, 136.5, 131.6, 128.4, 128.22 (d, ³J_{C-F}=8.7 Hz), 128.15, 125.5 (d, ⁵J_{C-F}=1.1 Hz), 125.4 (d, ³J_{C-F}=8.9 Hz), 125.2 (d, ⁴J_{C-F}=2.5 Hz), 117.4, 116.6 (d, ²J_{C-F}=25.4 Hz), 106.3 (d, ²J_{C-F}=22.4 Hz), 62.2, 61.7, 30.6, 12.7. ¹⁹F NMR (CDCl₃, 377 MHz): δ-114.04 (ddd, J=10.1, 8.4, 5.6 Hz). HRMS calculated for C₂₀H₁₈FN₂O₂[M+H]⁺: 337.134682. Found 337.135223.

### Preparation of 2-chloro-5-((7-fluoro-1,4-dimethoxy-3-methylnaphthalen-2-yl)methyl)pyrimidine (36)

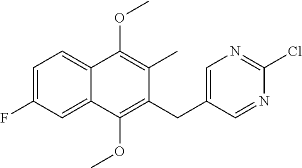 [SMILES: COc1c(C)c(Cc2cnc(Cl)nc2)c(OC)c2cc(F)ccc12]

This compound was isolated by purification by flash chromatography on silica gel (Cyclohexane/Ethyl acetate, 4/1, v/v, UV) with 70% yield (white solid). m.p.=139-141° C. ¹H NMR (CDCl₃, 400 MHz): δ8.41 (s, 2H), 8.06 (dd, J=9.2, 5.6 Hz, 1H), 7.63 (dd, J=10.2, 2.5 Hz, 1H), 7.26 (td, J=8.8, 2.6 Hz, 1H), 4.15 (s, 2H), 3.85 (s, 3H), 3.82 (s, 3H), 2.25 (s, 3H). ¹³C NMR (CDCl₃, 101 MHz): δ 161.1 (d, ¹J_{C-F}=246.2 Hz), 159.4, 159.3 (2C), 151.1 (d, ⁴J_{C-F}=1.3 Hz), 150.1 (d, ⁴J_{C-F}=5.4 Hz), 132.3, 128.2 (d, ³J_{C-F}=8.7 Hz), 127.8, 125.5 (d, ⁵J_{C-F}=0.5 Hz), 125.4 (d, ³J_{C-F}=8.9 Hz), 124.9 (d, ⁵J_{C-F}=2.5 Hz), 116.6 (d, ²J_{C-F}=25.4 Hz), 106.3 (d, ²J_{C-F}=22.4 Hz), 62.2, 61.7, 27.3, 12.7. ¹⁹F NMR (CDCl₃, 377 MHz): δ-113.94 (ddd, J=10.1, 8.4, 5.6 Hz). HRMS calculated for C₁₈H₁₇CIFN₂O₂[M+H]⁺: 347.095710. Found 347.097652.

### Preparation of 2-((7-fluoro-1,4-dimethoxy-3-methylnaphthalen-2-yl)methyl)furan (38)

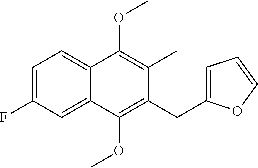 [SMILES: COc1c(C)c(Cc2ccco2)c(OC)c2cc(F)ccc12]

This compound were isolated by purification by flash chromatography on silica gel (Cyclohexane/Ethyl acetate, gradient from I/O to 99/1, v/v, UV) with 83% yield (colourless oil). ¹H NMR (CDCl₃, 400 MHz): δ 8.09 (dd, J=9.2, 5.6 Hz, 1H), 7.68 (dd, J=10.4, 2.6 Hz, 1H), 7.32 (br d, J=1.1 Hz, 1H), 7.26 (ddd, J=9.1, 8.3, 2.6 Hz, 1H), 6.26 (dd, J=3.1, 1.9 Hz, 1H), 5.87-5.82 (m, 1H), 4.23 (s, 2H), 3.87 (d, J=3.4 Hz, 6H), 2.36 (s, 3H). ¹³C NMR (CDCl₃, 101 MHz): δ 161.0 (d, ¹J_{C-F}=245.2 Hz), 154.0, 150.5 (d, ⁵J_{C-F}=1.4 Hz), 150.2 (d, ⁴J_{C-F}=5.4 Hz), 141.3, 128.4, 128.3 (d, ³J_{C-F}=8.7 Hz), 126.3 (d, ⁴J_{C-F}=2.4 Hz), 125.3, 125.2 (d, ³J_{C-F}=8.9 Hz), 116.2 (d, ²J_{C-F}=25.4 Hz), 110.4, 106.4 (d, ²J_{C-F}=22.3 Hz), 106.0, 62.5, 61.6, 26.5, 12.4. ¹⁹F NMR (CDCl₃, 377 MHz): δ-114.95 (ddd, J=10.4, 8.4, 5.7 Hz). HRMS calculated for C₁₈H₁₈FO₃ [M+H]⁺: 301.123449. Found 301.123811.

### Preparation of 5-((7-fluoro-1,4-dimethoxy-3-methylnaphthalen-2-yl)methyl)-2-(trifluoromethyl)pyrimidine (37)

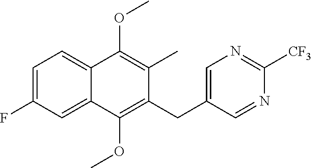 [SMILES: COc1c(C)c(Cc2cnc(C(F)(F)F)nc2)c(OC)c2cc(F)ccc12]

This compound was isolated by purification by flash chromatography on silica gel (Cyclohexane/Ethyl acetate, 4/1, v/v, UV) with 73% yield (colourless oil). ¹H NMR (CDCl₃, 400 MHz): δ 8.69 (s, 2H), 8.08 (dd, J=9.2, 5.6 Hz, 1H), 7.64 (dd, J=10.2, 2.5 Hz, 1H), 7.28 (ddd, J=9.1, 8.4, 2.6 Hz, 1H), 4.27 (s, 2H), 3.85 (d, J=12.1 Hz, 6H), 2.28 (s, 3H). ¹³C NMR (CDCl₃, 101 MHz): δ 161.2 (d, ¹J_{C-F}=246.3 Hz), 157.6 (2C), 154.9 (q, ²J_{C-F}=36.9 Hz), 151.2 (d, ⁵J_{C-F}=1.3 Hz), 150.2 (d, ⁴J_{C-F}=5.3 Hz), 136.1, 128.6 (d, ³J_{C-F}=8.7 Hz), 127.4, 125.7, 125.5 (d, ³J_{C-F}=8.9 Hz), 124.9 (d, ⁴J_{C-F}=2.5 Hz), 119.8 (d, ¹J_{C-F}=275.7 Hz), 116.8 (d, ²J_{C-F}=25.4 Hz), 106.4 (d, ²J_{C-F}=22.5 Hz), 62.2, 61.8, 28.1, 12.8. ¹⁹F NMR (CDC₃, 377 MHz): δ-70.12, −113.87 (ddd, J=10.1, 8.4, 5.6 Hz).

### Post-Modifications of Suzuki Coupling Products

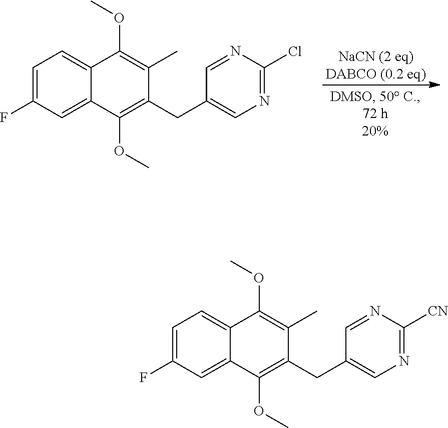 [SMILES: COc1c(C)c(Cc2cnc(Cl)nc2)c(OC)c2cc(F)ccc12 ; COc1c(C)c(Cc2cnc(C#N)nc2)c(OC)c2cc(F)ccc12]

### Preparation of 5-((7-fluoro-1,4-dimethoxy-3-methylnaphthalen-2-yl)methyl)pyrimidine-2-carbonitrile (40)

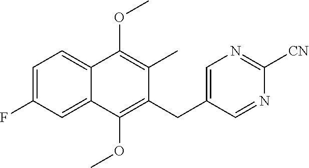 [SMILES: COc1c(C)c(Cc2cnc(C#N)nc2)c(OC)c2cc(F)ccc12]

A solution of 36 (100 mg, 0.29 mmol, 1 eq) in DMSO (1.0 mL) was added a solution of sodium cyanide (28.26 mg, 0.58 mmol, 2 eq) and DABCO (6.81 mg, 0.058 mmol, 0.2 eq) in DMSO (1 mL) and water (0.4 mL). The reaction mixture was stirred at 50° C. for 72 h. The reaction mixture was diluted with water and extracted twice with diethyl ether. Reunited organic layers were washed with brine and concentrated under reduced pressure. The crude residue was purified by flash chromatography on silica gel (Cyclohexane/Ethyl acetate, 4/1, v/v) to afford 40 as a white solid (19 mg, 20%). m.p.=167-169° C. ¹H NMR (CDCl₃, 400 MHz): δ 8.63 (s, 2H), 8.09 (dd, J=9.2, 5.6 Hz, 1H), 7.64 (dd, J=10.2, 2.5 Hz, 1H), 7.30 (ddd, J=9.1, 8.4, 2.6 Hz, 1H), 4.25 (s, 2H), 3.87 (s, 3H), 3.85 (s, 3H), 2.27 (s, 3H). ¹³C NMR (CDCl₃, 101 MHz): δ 161.3 (d, ¹J_{C-F}=246.7 Hz), 157.8 (2C), 151.3 (d, ⁵J_{C-F}=1.3 Hz), 150.3 (d, ⁴J_{C-F}=5.5 Hz), 143.2, 136.9, 128.3 (d, ³J_{C-F}=8.7 Hz), 127.1, 125.8, 125.5 (d, ³J_{C-F}=8.9 Hz), 124.7 (d, ⁴J_{C-F}=2.5 Hz), 117.0 (d, ²J_{C-F}=25.4 Hz), 115.8, 106.4 (d, ²J_{C-F}=22.5 Hz), 62.3, 61.9, 28.4, 12.9. ¹⁹F NMR (CDCl₃, 377 MHz): δ-113.65 (ddd, J=9.9, 8.4, 5.6 Hz). HRMS calculated for C₁₉H₁₇FN₃O₂[M+H]⁺: 338.129931. Found 338.131588.

### Preparation of Compounds of Formula (1) (by Oxidative Deprotection from the Compounds of Formula (11))

The compounds of formula (1) wherein R¹═F are prepared according to the following reaction scheme:

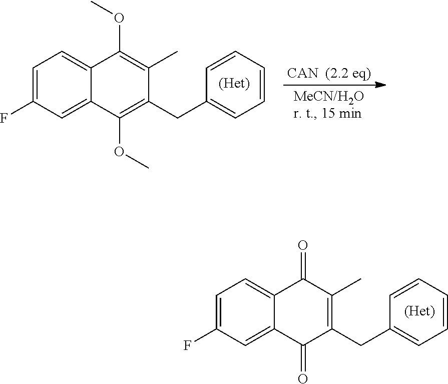

### General Procedure for the Oxidative Deprotection:

To a solution of 3-R-6-R′-1,4-dimethoxy-2-methylnaphetalene (1 eq) in acetonitrile was added dropwise a solution of ceric ammonium nitrate (2.2 eq) in water. The solution was stirred at room temperature for 15 min. The reaction mixture was diluted with water and extracted twice with dichloromethane. The organic layer was washed with brine, dried over MgSO₄ and concentrated under reduced pressure.

The crude residue was purified by flash chromatography on silica gel to afford the desired product.

| Compound of formula (I) | Yield |
|---|---|
| 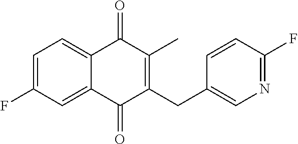 [SMILES: CC1=C(Cc2ccc(F)nc2)C(=O)c2cc(F)ccc2C1=O] | 71% |
| 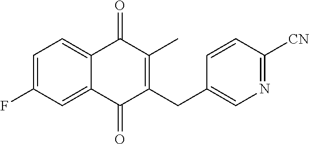 [SMILES: CC1=C(Cc2ccc(C#N)nc2)C(=O)c2cc(F)ccc2C1=O] | 85% |
| 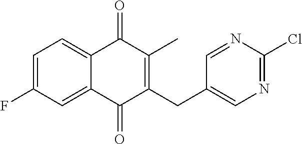 [SMILES: CC1=C(Cc2cnc(Cl)nc2)C(=O)c2cc(F)ccc2C1=O] | 72% |
| 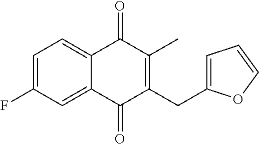 [SMILES: CC1=C(Cc2ccco2)C(=O)c2cc(F)ccc2C1=O] | 15% |
| 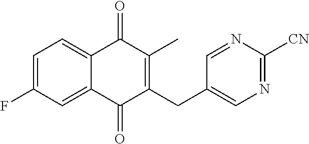 [SMILES: CC1=C(Cc2cnc(C#N)nc2)C(=O)c2cc(F)ccc2C1=O] | 75% |
| 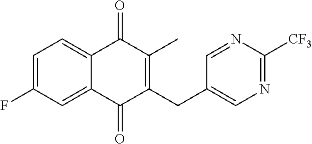 [SMILES: CC1=C(Cc2cnc(C(F)(F)F)nc2)C(=O)c2cc(F)ccc2C1=O] | 96% |

### Preparation of 6-fluoro-3-((6-fluoropyridin-3-yl)methyl)-2-methylnaphthalene-1,4-dione (14)

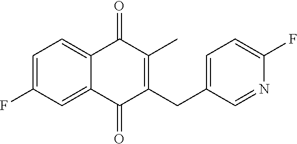 [SMILES: CC1=C(Cc2ccc(F)nc2)C(=O)c2cc(F)ccc2C1=O]

0 4 was isolated by purification by flash chromatography on silica gel (Cyclohexane/Ethyl acetate, gradient from 4/1 to 7/3, v/v, UV) with 71% yield (yellow solid). m.p.=141-143° C. ¹H NMR (CDCl₃, 400 MHz): δ8.12 (dd, J=8.6, 5.1 Hz, 2H), 7.70 (dd, J=8.5, 2.6 Hz, 1H), 7.66 (dd, J=8.1, 2.4 Hz, 1H), 7.36 (td, J=8.4, 2.6 Hz, 1H), 6.83 (dd, J=8.4, 3.0 Hz, 1H), 3.98 (s, 2H), 2.27 (s, 3H). ¹³C NMR (CDCl₃, 101 MHz): δ 183.8, 183.5 (d, ⁴J_{C-F}=1.4 Hz), 166.2 (d, ¹J_{C-F}=257.3 Hz), 162.6 (d, ¹J_{C-F}=238.5 Hz), 147.4 (d, ³J_{C-F}=14.6 Hz), 145.1, 144.3 (d, ⁵J_{C-F}=1.0 Hz), 141.5 (d, ³J_{C-F}=7.8 Hz), 134.5 (d, ³J_{C-F}=7.9 Hz), 131.3 (d, ⁴J_{C-F}=4.6 Hz), 129.9 (d, ³J_{C-F}=8.9 Hz), 128.7 (d, ⁴J_{C-F}=3.3 Hz), 121.1 (d, ²J_{C-F}=22.6 Hz), 113.3 (d, ²J_{C-F}=23.5 Hz), 109.7 (d, ²J_{C-F}=37.5 Hz), 29.2, 13.5. ¹⁹F NMR (CDCl₃, 377 MHz): δ-70.7 (m), −102.03 (td, J=8.3, 5.3 Hz). HRMS calculated for C₁₇H₁₂F₂NO₂ [M+H]⁺: 300.083061. Found 300.083945.

### Preparation of 5-((7-fluoro-3-methyl-1,4-dioxo-1,4-dihydronaphthalen-2-yl)methyl)picolinonitrile (15)

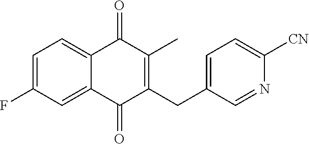 [SMILES: CC1=C(Cc2ccc(C#N)nc2)C(=O)c2cc(F)ccc2C1=O]

0 15 was isolated by purification by flash chromatography on silica gel (Cyclohexane/Ethyl acetate, gradient from 4/1 to 7/3, v/v, UV) with 85% yield (yellow solid). m.p.=153-154° C. ¹H NMR (CDCl₃, 400 MHz): δ 8.62 (d, J=1.6 Hz, 1H), 8.11 (dd, J=8.6, 5.2 Hz, 1H), 7.68 (td, J=8.4, 7.6, 2.3 Hz, 2H), 7.60 (d, J=8.0 Hz, 1H), 7.37 (td, J=8.3, 2.6 Hz, 1H), 4.07 (s, 2H), 2.27 (s, 3H). ¹³C NMR (CDCl₃, 101 MHz): δ 183.4, 183.3 (d, ⁴J_{C-F}=1.5 Hz), 166.1 (d, ¹J_{C-F}=257.6 Hz), 151.5, 145.7, 143.2 (d, ⁴J_{C-F}=1.9 Hz), 138.1, 137.1, 134.3 (d, ³J_{C-F}=8.0 Hz), 132.1, 130.0 (d, ³J_{C-F}=8.9 Hz), 128.7 (d, ⁴J_{C-F}=3.3 Hz), 128.5, 121.2 (d, ²J_{C-F}=22.5 Hz), 117.2, 113.3 (d, ²J_{C-F}=23.5 Hz), 30.3, 13.6. ¹⁹F NMR (CDCl₃, 377 MHz): δ-101.69 (td, J=8.2, 5.3 Hz). HRMS calculated for C₁₈H₁₂FN₂O₂ [M+H]⁺: 307.087732. Found 307.088742.

### Preparation of 6-fluoro-3-(furan-2-ylmethyl)-2-methylnaphthalene-1,4-dione (18)

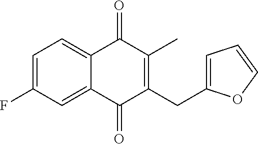 [SMILES: CC1=C(Cc2ccco2)C(=O)c2cc(F)ccc2C1=O]

18 was isolated by purification by flash chromatography on silica gel (Cyclohexane/Ethyl acetate, gradient from 4/1 to 7/3, v/v, UV) with 14% yield (sticky yellow dark solid). ¹H NMR (CDCl₃, 400 MHz): δ 8.12 (dd, J=8.6, 5.3 Hz, 1 H), 7.73 (dd, J=8.6, 2.6 Hz, 1H), 7.35 (td, J=8.3, 2.7 Hz, 1H), 7.27 (dd, J=1.8, 0.7 Hz, 1H), 6.27 (dd, J=3.2, 1.9 Hz, 1H), 6.08 (dd, J=3.2, 0.8 Hz, 1H), 4.03 (s, 2H), 2.29 (s, 3H). ¹³C NMR (CDCl₃, 101 MHz): δ 184.1, 183.0 (d, ⁴J_{C-F}=1.4 Hz), 166.1 (d, ¹J_{C-F}=256.8 Hz), 151.0, 145.4, 142.6 (d, ⁵J_{C-F}=2.0 Hz), 141.7, 134.7 (d, ³J_{C-F}=7.9 Hz), 129.8 (d, ³J_{C-F}=8.9 Hz), 128.9 (d, ⁴J_{C-F}=3.3 Hz), 120.8 (d, ²J_{C-F}=22.6 Hz), 113.3 (d, ²J_{C-F}=23.5 Hz), 110.6, 106.9, 25.7, 13.2. ¹⁹F NMR (CDCl₃, 377 MHz): δ-102.49 (td, J=8.3, 5.3 Hz). HRMS calculated for C₁₆H₁₂FO₃ [M+H]⁺: 271.076499. Found 271.078115.

### Preparation of 3-((2-chloropyrimidin-5-yl)methyl)-6-fluoro-2-methylnaphthalene-1,4-dione (16)

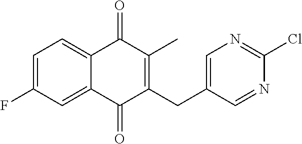 [SMILES: CC1=C(Cc2cnc(Cl)nc2)C(=O)c2cc(F)ccc2C1=O]

16 was isolated by purification by flash chromatography on silica gel (Cyclohexane/Ethyl acetate, 4/1, v/v, UV) with 72% yield (yellow solid). m.p.=167-168° C. ¹H NMR (CDCl₃, 400 MHz): δ 8.53 (s, 2H), 8.13 (dd, J=8.6, 5.2 Hz, 1H), 7.69 (dd, J=8.5, 2.6 Hz, 1H), 7.38 (td, J=8.3, 2.6 Hz, 1H), 3.96 (s, 2H), 2.30 (s, 3H). ¹³C NMR (CDCl₃, 101 MHz): δ 183.4, 183.2 (d, ⁴J_{C-F}=1.5 Hz), 166.2 (d, ¹J_{C-F}=258.6 Hz), 160.0, 159.7 (2C), 145.5, 142.9 (d, ⁵J_{C-F}=1.9 Hz), 134.3 (d, ³J_{C-F}=7.9 Hz), 130.4, 130.0 (d, ³J_{C-F}=8.9 Hz), 128.6 (d, ⁴J_{C-F}=3.3 Hz), 121.3 (d, ²J_{C-F}=22.5 Hz), 113.4 (d, ²J_{C-F}=23.6 Hz), 27.2, 13.7. ¹⁹F NMR (CDCl₃, 377 MHz): δ -101.54 (td, J=8.2, 5.2 Hz). HRMS calculated for C₁₆H₁₁CIFN₂O₂[M+H]⁺: 317.048760. Found 317.049497.

### Preparation of 5-((7-fluoro-3-methyl-1,4-dioxo-1,4-dihydronaphthalen-2-yl)methyl)pyrimidine-2-carbonitrile (20)

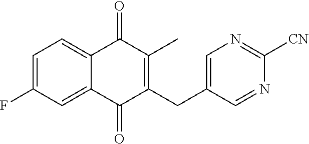 [SMILES: CC1=C(Cc2cnc(C#N)nc2)C(=O)c2cc(F)ccc2C1=O]

20 was isolated by purification by flash chromatography on silica gel (Cyclohexane/Ethyl acetate, 4/1, v/v, UV) with 75% (yellow solid). m.p.=187-188° C. ¹H NMR (CDCl₃, 400 MHz): δ 8.76 (s, 2H), 8.15 (dd, J=8.6, 5.2 Hz, 1H), 7.71 (dd, J=8.4, 2.6 Hz, 1H), 7.41 (td, J=8.3, 2.6 Hz, 1H), 4.06 (s, 2H), 2.33 (s, 3H). ¹³C NMR (CDCl₃, 101 MHz): δ 183.20, 183.18, 166.3 (d, ¹J_{C-F}=258.1 Hz), 158.1 (2C), 146.0, 143.6, 142.2, 134.9, 134.2 (d, ³J_{C-F}=7.9 Hz), 130.2 (d, ³J_{C-F}=8.9 Hz), 128.7 (d, ⁴J_{C-F}=3.3 Hz), 121.5 (d, ²J_{C-F}=22.6 Hz), 115.7, 113.5 (d, ²J_{C-F}=23.6 Hz), 28.3, 13.8. ¹⁹F NMR (CDCl₃, 377 MHz): δ -101.19 (td, J=8.2, 5.3 Hz). HRMS calculated for C₁₇H₁₁FN₃O₂[M+H]⁺: 308.082981. Found 308.084056.

### Preparation of 6-fluoro-2-methyl-3-((2-(trifluoromethyl)pyrimidin-5-yl)methyl)naphthalene-1,4-dione (17)

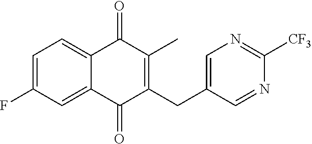 [SMILES: CC1=C(Cc2cnc(C(F)(F)F)nc2)C(=O)c2cc(F)ccc2C1=O]

17 was isolated by purification by flash chromatography on silica gel (Cyclohexane/Ethyl acetate, 7/3, v/v, UV) with 96% yield (yellow solid). m.p.=195-197° C. ¹H NMR (CDCl₃, 400 MHz): δ 8.81 (s, 2H), 8.14 (dd, J=8.6, 5.2 Hz, 1H), 7.70 (dd, J=8.4, 2.6 Hz, 1H), 7.39 (td, J=8.3, 2.7 Hz, 1H), 4.07 (s, 2H), 2.33 (s, 3H). ¹³C NMR (CDCl₃, 101 MHz): δ 183.3, 183.2 (d, ⁴J_{C-F}=1.4 Hz), 166.3 (d, ¹J_{C-F}=257.9 Hz), 158.0, 155.4 (q, ²J_{C-F}=37.1 Hz), 145.8, 142.6 (d, ⁵J_{C-F}=1.8 Hz), 134.3 (d, ³J_{C-F}=8.2 Hz), 134.2, 130.1 (d, ³J_{C-F}=8.9 Hz), 128.7 (d, ⁴J_{C-F}=3.3 Hz), 121.4 (d, ²J_{C-F}=22.5 Hz), 119.6 (d, ¹J_{C-F}=275.3 Hz), 113.5 (d, ²J_{C-F}=23.6 Hz), 28.0, 13.7. ¹⁹F NMR (CDCl₃, 377 MHz): δ-70.24, −101.44 (td, J=8.2, 5.3 Hz). HRMS calculated for C₁₇H₁₁F₄N₂O₂ [M+H]⁺: 351.075117. Found 351.075663.

### Preparation of 6-fluoro-2-methyl-3-(thiophen-2-ylmethyl)naphthalene-1,4-dione (19)

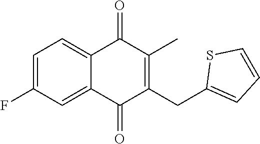 [SMILES: CC1=C(Cc2cccs2)C(=O)c2cc(F)ccc2C1=O]

M.p.=60-62° C. ¹H NMR (CDCl₃, 400 MHz): δ8.10 (dd, J=8.6, 5.3 Hz, 1H), 7.73 (dd, J=8.6, 2.6 Hz, 1H), 7.34 (td, J=8.3, 2.7 Hz, 1H), 7.11 (dd, J=4.9, 1.4 Hz, 1H), 6.92-6.85 (m, 2H), 4.17 (s, 2H), 2.29 (s, 3H). ¹³C NMR (CDCl₃, 101 MHz): δ 184.1, 183.2 (d, ⁴J_{C-F}=1.4 Hz), 166.1 (d, ¹J_{C-F}=256.9 Hz), 144.6 (d, ⁵J_{C-F}=1.9 Hz), 144.4, 139.7, 134.6 (d, ³J_{C-F}=7.9 Hz), 129.7 (d, ³J_{C-F}=8.8 Hz), 128.8 (d, ⁴J_{C-F}=3.2 Hz), 127.0, 125.9, 124.3, 120.8 (d, ²J_{C-F}=22.6 Hz), 113.3 (d, ²J_{C-F}=23.2 Hz), 27.1, 13.2. ¹⁹F NMR (CDCl₃, 377 MHz): δ-102.36 (td, J=8.3, 5.3 Hz). HRMS calculated for C₁₆H₁₂FO₂S [M+H]⁺: 287.053655. Found 287.054700.

### COMPARATIVE EXAMPLE

It was shown that the order of the steps is essential for the process of the invention for the preparation of the intermediate compounds of formula (IV).

The following reaction was carried out:

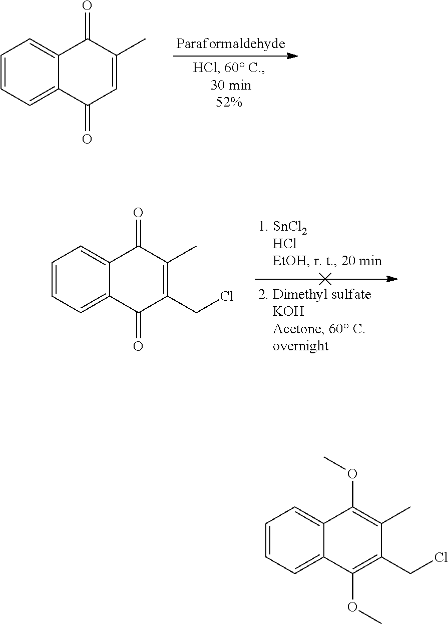

By carring out a chloromethylation step from menadione, the compound of formula (IV) could not be obtained.

Moreover, the yield for this chloromethylation step was lower in comparison with the yield for this step in the preparation of compounds of formula (IV) from compounds of formula (V).

### Comparative Biological Results

Regioisomeric compounds were synthetized using the process of WO 2020/252414 based on organozinc intermediates. MR00397 and MR00407 were obtained with very low yields (Scheme 2, Table):

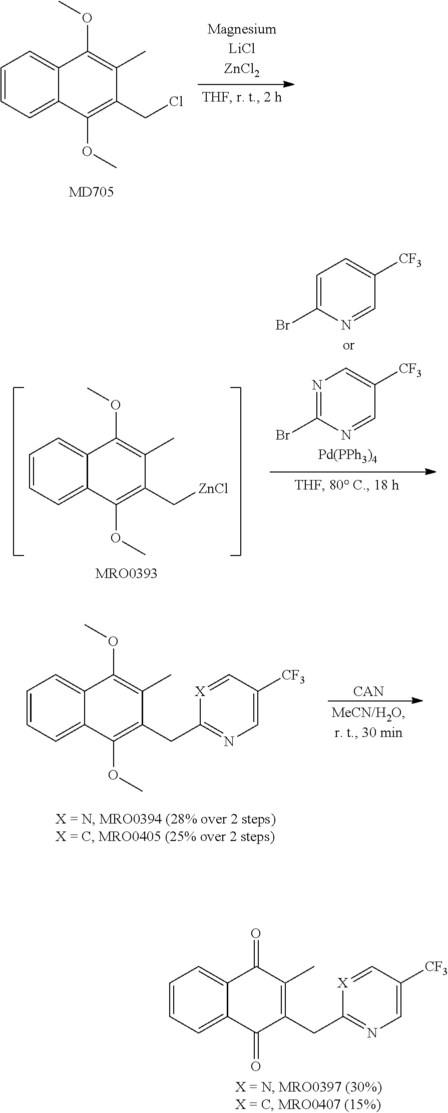

The synthesis of pyridinyl/pyrimidinyl derivatives of formula (I) according to the invention was investigated using the same method. Unfortunately, both molecules were obtained in inseparable mixtures. The mixtures were allowed to react with CAN. MR00418 was isolated with a second fluorinated compound and MR00419 with 4 other fluorinated compounds. The structures of the (non separated) side-products were not elucidated.

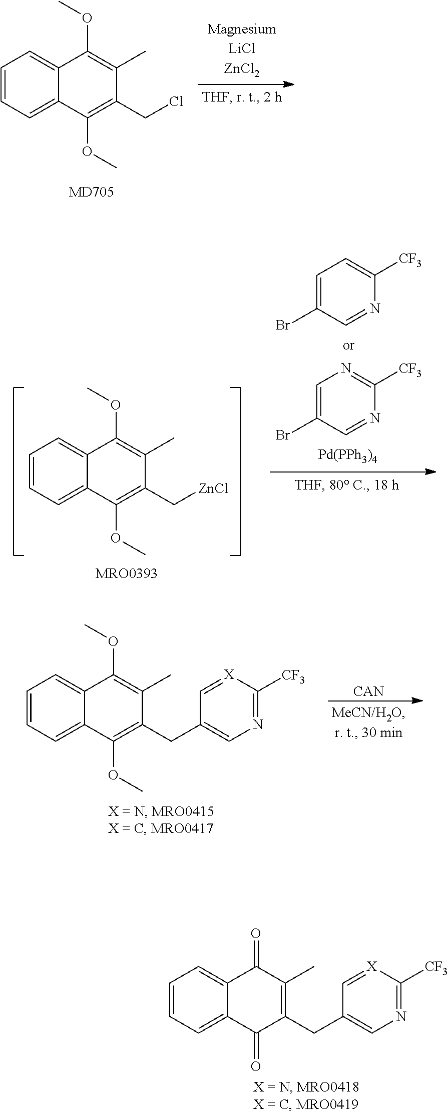

The method according to the invention is thus the most efficient synthetic pathway to synthetize MR00418 and MR00419. With this method, MR00418 was obtained with total 84% yield in 2 steps from MD705. MR00419 was synthetized in 2 steps from MD705 with total 67% yield in 2 steps

The first primary screening, performed with the Plasmodium falciparum NF54 strain and the rat L6 myoblast cell line, showed that both MR00418 and MR00419 displayed more potent and specific antimalarial activities than the regioisomers MR00397 and MR00407, respectively:

| | NF54 IC50 | L6 IC50 |
|---|---|---|
| | (nM) | (μM) |
| MRO0418 (invention) | 6 | 179.3 |
| MRO0397 | 567 | N.D. |
| MRO0419 (invention) | 65 | 48.3 |
| MRO0407 | 84 | N.D. |
| plasmodione | 8 | 141.7 |

What is claimed is:

1-15. (canceled)

16. A compound having the formula (I): 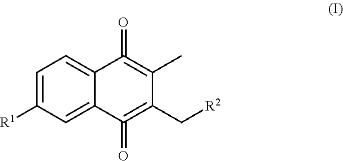
wherein:
R¹ is selected from the group consisting of: H, F, (C₁-C₆)alkoxy, and halo(C₁-C₆)alkyl; and
R² is a heteroaryl group, said heteroaryl group being different from the following heteroaryl groups: 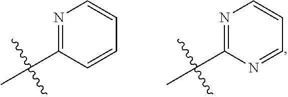 [SMILES: *c1ccccn1 ; *c1ncccn1]
provided that the compound of formula (I) is different from the following compound:

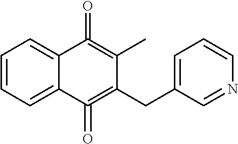

17. The compound according to claim 16, wherein the heteroaryl group is substituted with at least one substituent selected from the group consisting of:
halogen,
halo(C₁-C₆)alkyl,
(C₁-C₆)alkoxy,
CN,
(C₁-C₆)alkyl,
NO₂,
NRₐR_{b}, Rₐ and R_{b}, identical or different, being independently H or a (C₁-C₆)alkyl, such as NH₂,
(C₂-C₆)alkynyl, such as —C≡C—,
OR, R_{c} being a cycloheteroalkyl, preferably an oxetanyl group,
SF₃,
SF₅,
—C(═O)—(C₁-C₆)alkyl,
halo(C₁-C₆)alkoxy, and
(C1-C6)alkoxy.

18. The compound according to claim 16, wherein R² is a heteroaryl group comprising a 5- to 10-membered aromatic monocyclic or bicyclic group containing from 1 to 4 heteroatoms selected from O, S or N.

19. The compound according to claim 16, wherein R¹ is H or F and/or R² is selected from the group consisting of: pyridinyl other than 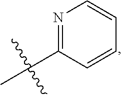 [SMILES: *c1ccccn1]
pyrimidinyl other than 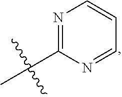 [SMILES: *c1ncccn1]
quinolinyl, thiophenyl, and furanyl groups.

20. The compound according to claim 19, wherein R² is selected from the group consisting of: pyridinyl other than 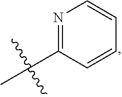 [SMILES: *c1ccccn1]
pyrimidinyl other than 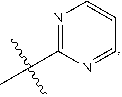 [SMILES: *c1ncccn1]
quinolinyl, thiophenyl, and furanyl groups, said groups being substituted with at least one substituent selected from the group consisting of: halogen, amino, halo(C₁-C₆)alkyl, (C₁-C₆)alkoxy, —C≡C—, and CN.

21. The compound according to claim 16, being selected from the following compounds:

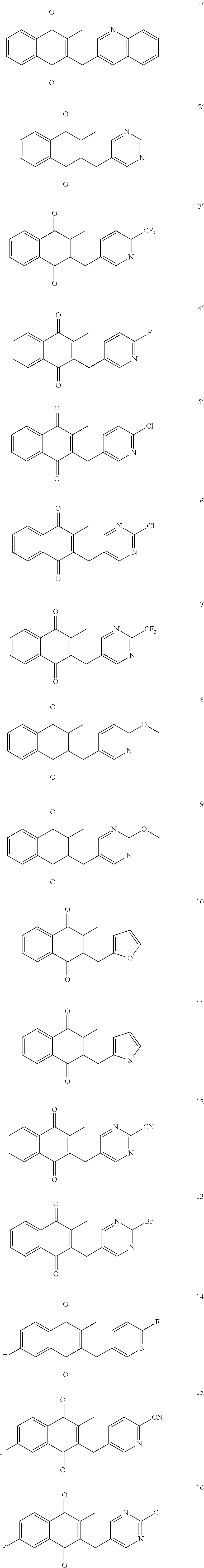

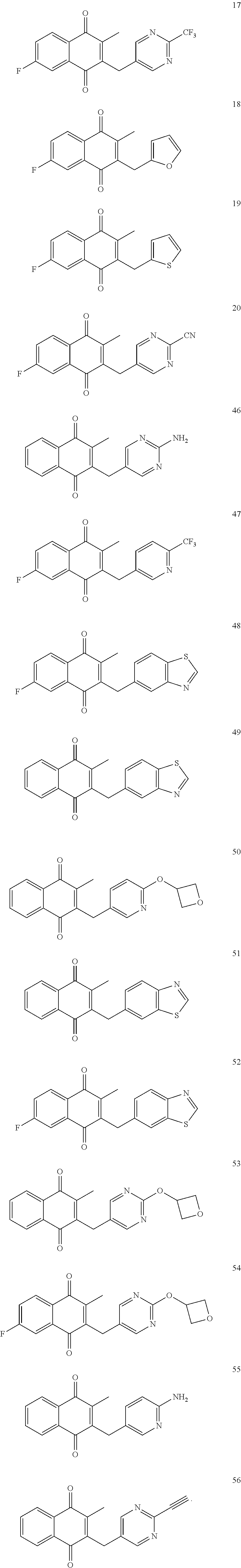

22. A process for the preparation of a compound having the formula (1): 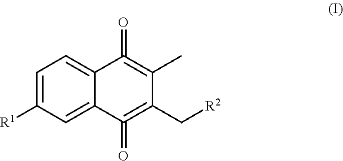
wherein:
R¹ is selected from the group consisting of: H, F, (C₁-C₆)alkoxy, and halo(C₁-C₆)alkyl; and
R² is a heteroaryl group, said heteroaryl group being different from the following heteroaryl groups: 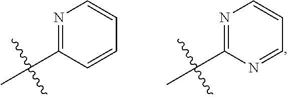 [SMILES: *c1ccccn1 ; *c1ncccn1]
said process comprising the preparation of a compound having the following formula (IV):
wherein: 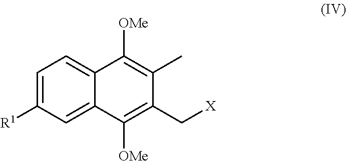
R¹ is selected from the group consisting of: H, F, (C₁-C₆)alkoxy, and halo(C₁-C₆)alkyl; and
X is Cl or Br,
by the chloromethylation or bromomethylation of a compound having the following formula (V): 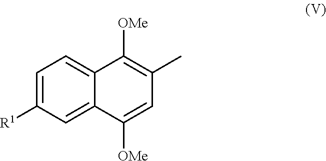
wherein R¹ is selected from the group consisting of: H, F, (C₁-C₆)alkoxy, and halo(C₁-C₆)alkyl.

23. The process according to claim 22, wherein the heteroaryl group is substituted with at least one substituent selected from the group consisting of:
halogen,
halo(C₁-C₆)alkyl,
(C₁-C₆)alkoxy,
CN,
(C₁-C₆)alkyl,
NRₐR_{b}, Rₐ and R_{b}, identical or different, being independently H or a (C₁-C₆)alkyl, such as NH₂,
(C₂-C₆)alkynyl, such as —C≡C—,
OR, R being a cycloheteroalkyl, preferably an oxetanyl group,
NO₂,
SF₃,
SF₅,
—C(═O)—(C₁-C₆)alkyl,
halo(C₁-C₆)alkoxy, and
(C₁-C₆)alkoxy.

24. The process according to claim 22, wherein the chloromethylation or bromomethylation step is carried out with a mixture of hydrochloric acid or hydrobromic acid with paraformaldehyde in the presence of a solvent selected from the group consisting of: water, acetic acid, and dioxane.

25. The process according to claim 22, further comprising the pallado-catalyzed Suzuki coupling of the compound of formula (IV) with a boronic acid compound having the formula (III) or (III′): 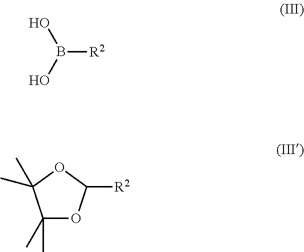
wherein R² is selected from the group consisting of: pyridinyl other than 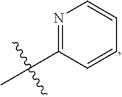 [SMILES: *c1ccccn1]
pyrimidinyl other than 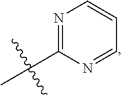 [SMILES: *c1ncccn1]
quinolinyl, thiophenyl, and furanyl groups in order to obtain a compound having the following formula (II): 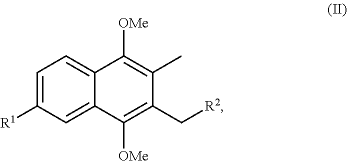
wherein R¹ is H or F and/or R² is selected from the group consisting of: pyridinyl other than 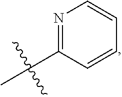 [SMILES: *c1ccccn1]
pyrimidinyl other than 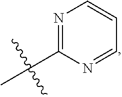 [SMILES: *c1ncccn1]
quinolinyl, thiophenyl, and furanyl groups.

26. The process according to claim 25, wherein the pallado-catalyzed coupling is carried out with a palladium catalyst selected from the group consisting of: Pd(PPh₃)₄, PdCl₂, PdCl₂(dppf), Pd(OAc)₂ and PPh₃, and with a base selected from the group consisting of: Na₂CO₃, K₂CO₃, KOtBu, Cs₂CO₃, NaOH, and NEt₃, or with K₃PO₄ in toluene.

27. The process according to claim 25, further comprising an oxidative demethylation step of the compound of formula (II) in the presence of an oxidant, in order to obtain the compound of formula (1).

28. The process according to claim 27, wherein the oxidant is selected from the group consisting of: ceric ammonium nitrate, silver oxide (AgO/Ag₂O), OsO₄/NalO₄, oxone, BBr₃ with O₂ or open air, and boron trichloride/tetra-n-butylammonium iodide (BCl₃/TBAI) with O₂ or open air.

29. The process according to claim 22, wherein the compound of formula (V) is prepared by reacting a compound having the following formula (VI): 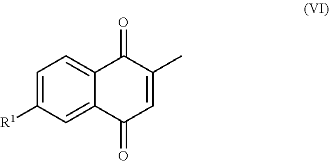
wherein R¹ is selected from the group consisting of: H, F, (C₁-C₆)alkoxy, and halo(C₁-C₆)alkyl,
with a reducing agent, in particular being SnCl₂ and HCl, or sodium dithionite, followed by a methylation step, in order to obtain the compound of formula (V).

30. The process according to claim 22, wherein R² is selected from the group consisting of: pyridinyl other than 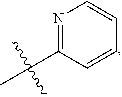 [SMILES: *c1ccccn1]
pyrimidinyl other than 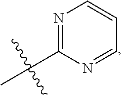 [SMILES: *c1ncccn1]
quinolinyl, thiophenyl, and furanyl groups.

31. The process according to claim 30, wherein R² is selected from the group consisting of: pyridinyl other than 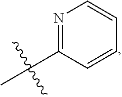 [SMILES: *c1ccccn1]
pyrimidinyl other than 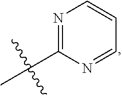 [SMILES: *c1ncccn1]
quinolinyl, thiophenyl, and furanyl groups, said groups being substituted with at least one substituent selected from the group consisting of: halogen, amino, halo(C₁-C₆)alkyl, (C₁-C₆)alkoxy, —C≡C—, and CN.

32. A compound having the following formula (II): 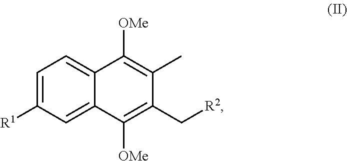
wherein:
R¹ is selected from the group consisting of: H, F, (C₁-C₆)alkoxy, and halo(C₁-C₆)alkyl; and
R² is a heteroaryl group, said heteroaryl group being different from the following heteroaryl groups: 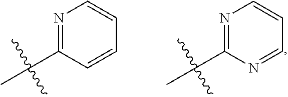 [SMILES: *c1ccccn1 ; *c1ncccn1]
provided that the compound of formula (II) is different from the following compound:

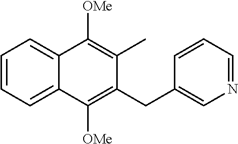 [SMILES: COc1c(C)c(Cc2cccnc2)c(OC)c2ccccc12]

33. The compound according to claim 32, wherein the heteroaryl group is substituted with at least one substituent selected from the group consisting of:
halogen,
halo(C₁-C₆)alkyl,
(C₁-C₆)alkoxy,
CN,
(C₁-C₆)alkyl,
NRₐR_{b}, Rₐ and R_{b}, identical or different, being independently H or a (C₁-C₆)alkyl, such as NH₂,
(C₂-C₆)alkynyl, such as —C≡C—,
OR, R_{c} being a cycloheteroalkyl, preferably an oxetanyl group,
NO₂,
SF₃,
SF₅,
—C(═O)—(C₁-C₆)alkyl,
halo(C₁-C₆)alkoxy, and
(C₁-C₆)alkoxy.

34. A compound having the following formula (IV): 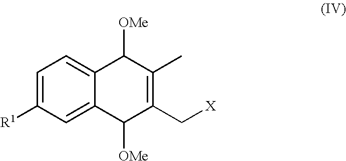
wherein R¹ is selected from the group consisting of: H, F, (C₁-C₆)alkoxy, and halo(C₁-C₆)alkyl,
provided that the compound of formula (IV) is different from the following compounds:

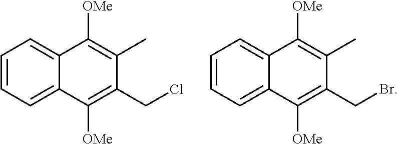

35. A compound having the following formula (V): 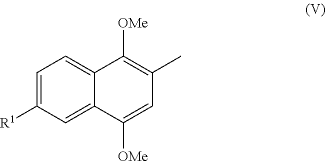
wherein R¹ is selected from the group consisting of: F, (C₁-C₆)alkoxy, and halo(C₁-C₆)alkyl.
